(19) United States
(12) Patent Application Publication
(10) Pub. No.: US 2025/0312440 A1
(43) Pub. Date: Oct. 9, 2025

(54) CHEMICAL SCREEN OF MODULATORS FOR VACCINE ADJUVANTS

(71) Applicant: THE UNIVERSITY OF CHICAGO, Chicago, IL (US)

(72) Inventors: Aaron ESSER-KAHN, Chicago, IL (US); Matthew ROSENBERGER, Chicago, IL (US); Jeremiah KIM, Chicago, IL (US)

(73) Assignee: THE UNIVERSITY OF CHICAGO, Chicago, IL (US)

(21) Appl. No.: 18/869,655

(22) PCT Filed: May 31, 2023
(86) PCT No.: PCT/US2023/067719

§ 371 Date: Nov. 26, 2024

Related U.S. Application Data
(60) Provisional application No. 63/347,438, filed on May 31, 2022.
(60) Provisional application No. 63/359,620, filed on Jul. 8, 2022.
(60) Provisional application No. 63/424,201, filed on Nov. 10, 2022.

(51) Int. Cl. A61K 39/215; A61K 31/166; A61K 31/4406; A61K 31/4439; A61K 31/444; A61K 31/4706; A61K 31/506; A61K 31/519; A61K 31/5377; A61K 31/5415; A61K 39/00; A61K 39/39; A61P 31/14; C12N 7/00; G01N 33/50; G01N 33/68

(52) U.S. Cl. CPC A61K 39/215; A61K 31/166; A61K 31/4406; A61K 31/4439; A61K 31/444; A61K 31/4706; A61K 31/506; A61K 31/519; A61K 31/5377; A61K 31/5415; A61K 39/39; A61P 31/14; C12N 7/00; G01N 33/5023; G01N 33/6866; G01N 33/6869; A61K 2039/541; A61K 2039/55516; A61K 2039/55522; A61K 2039/55561; C12N 2770/20034

(57) ABSTRACT

The disclosure provides for methods and compositions that can be used for modulating an immune response. Described are methods comprising administering an effective amount of (a) an adjuvant; and (b) one or more of bafetinib (INNO-406), LY3009120, MK-8353 (SCH900353), amodiaquine, zanubrutinib (BOB-3111), Ku55933, Tucidinostat, PD318088, WNK463, and TRx0237 (LMTX) mesylate to a subject. The methods may he for the vaccination of subjects, or for the treatment or prevention of cancer, graft rejection, graft versus host disease, a bacterial infection, or a viral infection in a subject. The method may be for modulating an immune response in vivo, in vitro, or ex vivo. The methods also include immune activation of a population of immune cells in vitro or ex vivo. Also described is a method for identifying the efficacy of an adjuvant.

### CROSS-REFERENCE TO RELATED APPLICATIONS

This application claims the benefit of priority to U.S. Provisional Patent Application Ser. No. 63/347,438, filed May 31, 2022, U.S. Provisional Patent Application Ser. No. 63/359,620, filed Jul. 8, 2022, and U.S. Provisional Patent Application Ser. No. 63/424,201, filed Nov. 10, 2022, each of which are hereby incorporated by reference in their entirety.

### STATEMENT REGARDING FEDERALLY SPONSORED RESEARCH OR DEVELOPMENT

This invention was made with government support under grant numbers 75N93019C00041-P00004-9999-1, 75N93019C00041-P00001-9999-1, 75N93019C00041-P00002-9999-1, 75N93019C00041-P00003-9999-1, 75N93019C00041-P00001-9999-2, AI124286, AI112194, GM099594, and HDTRA1-18-1-0052 awarded by the National Institutes of Health. The government has certain rights in the invention.

### BACKGROUND

### 1. Field of the Invention

The invention relates generally to the fields of immunology and immunotherapy. Described herein are methods and compositions that increase the safety and effectiveness of vaccines and immunotherapeutics.

### 2. Background

Toll-like receptor (TLR) activation is linked to the high immunogenicity and protective effects of vaccines.^{1,2} The incorporation of TLR adjuvants in sub-unit and epitope-based vaccine formulations has led to great improvements in both antibody and T-cell levels and antigen specificity.^{3,4} Currently, many small molecule adjuvants have been discovered.^{5,6} However, their tolerability in preclinical and clinical studies have limited the use of many of these compounds requiring either reformulation or redesign.⁷

Historically, discovery of adjuvants has been empirical, but with synthetic small molecule adjuvants, modern drug discovery techniques have been applied to optimize adjuvanticity. This has led to the development of a class of adjuvants collectively referred to as small molecule immune potentiators (SMIPs).^{8,9} In this class, imidazoquinolinones that activate toll-like receptor-7 and toll-like receptor-8 (TLR 7/8) such as imiquimod (R837) and resiquimod (R848) have been extensively studied. Imiquimod is currently approved for clinical immunotherapy use in topical creams.⁹⁻¹¹ These SMIPs have been shown to elicit antigen specific cellular responses when administered as adjuvants.¹²⁻¹⁴ Additionally, activation of TLR7/8 by resiquimod can lead to antitumor activity facilitated by APC activation of CD⁸⁺ T cells and CD⁴⁺ Th cells due to to IFN-γ, IL-2, and IL-10 production and hence enhanced proliferation.¹⁵⁻¹⁷ However, the high bioavailability of imidazoquinolinone and structurally-related compounds results in unacceptable levels of systemic inflammation due to adjuvant toxicity, greatly limiting their use.

Accordingly, current vaccination and immunotherapeutic methods, particularly those comprising the use of adjuvants, may pose safety concerns, and there is a need in the art for strategies for increasing the safety and tolerability of vaccines and cancer immunotherapeutics.

### SUMMARY OF THE INVENTION

The disclosure provides for methods and compositions that can be used for modulating an immune response. Described are methods comprising administering an effective amount of (a) an adjuvant; and (b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate (or any combination thereof) to a subject. The methods may be for the vaccination of a subject, or for the treatment or prevention of cancer, graft rejection, graft versus host disease, a bacterial infection, or a viral infection in a subject. The method may be for modulating an immune response in vivo, in vitro, or ex vivo. The methods also include immune activation of a population of immune cells in vitro or ex vivo. Also described is a method for identifying the efficacy of an adjuvant, the method comprising: (a) administering the adjuvant to a population of cells; (b) administering a PRR agonist to the population of cells; and (c) measuring expression of one or more cytokines from the cells. Also described are pharmaceutical compositions comprising: (a) an adjuvant; and (b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate (or a combination thereof). It is contemplated that 1, 2, 3, 4, 5, 6, 7, 8 or 9 of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate may be included in any methods or compositions described herein. In some aspects, 1, 2, 3, 4, 5, 6, 7 or 8 of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate may be excluded.

The method may comprise or further comprise administering an antigen to the subject. The pharmaceutical compositions may comprise an antigen. The antigen may be one that is associated with a cancer, bacteria, or virus. The antigen may be an antigen associated with a cancer, bacteria, or virus that the subject has, has been diagnosed with, and/or has symptoms of. The antigen may be an antigen associated with a cancer, bacteria, or virus that the subject is at risk for. The antigen may be a bacterial antigen, a viral antigen, or a tumor antigen. The antigen may be an antigen described herein.

The methods may comprise or consist of administering an adjuvant and bafetinib (INNO-406). The methods may comprise or consist of administering an adjuvant, bafetinib (INNO-406), and an antigen. The pharmaceutical composition may comprise or consist of an adjuvant and bafetinib (INNO-406). The pharmaceutical composition may comprise or consist of an adjuvant, bafetinib (INNO-406), and an antigen. The methods may comprise or consist of administering an adjuvant and LY3009120. The methods may comprise or consist of administering an adjuvant, LY3009120, and an antigen. The pharmaceutical composition may comprise or consist of an adjuvant and LY3009120. The pharmaceutical composition may comprise or consist of an adjuvant, LY3009120, and an antigen. The methods may comprise or consist of administering an adjuvant and MK-8353 (SCH900353). The methods may comprise or consist of administering an adjuvant, MK-8353 (SCH900353), and an antigen. The pharmaceutical composition may comprise or consist of an adjuvant and MK-8353 (SCH900353). The pharmaceutical composition may comprise or consist of an adjuvant, MK-8353 (SCH900353), and an antigen. The methods may comprise or consist of administering an adjuvant and amodiaquine. The methods may comprise or consist of administering an adjuvant, amodiaquine, and an antigen. The pharmaceutical composition may comprise or consist of an adjuvant and amodiaquine. The pharmaceutical composition may comprise or consist of an adjuvant, amodiaquine, and an antigen. The methods may comprise or consist of administering an adjuvant and zanubrutinib. The methods may comprise or consist of administering an adjuvant, zanubrutinib, and an antigen. The pharmaceutical composition may comprise or consist of an adjuvant and zanubrutinib. The pharmaceutical composition may comprise or consist of an adjuvant, zanubrutinib, and an antigen. The methods may comprise or consist of administering an adjuvant and Ku55933. The methods may comprise or consist of administering an adjuvant, Ku55933, and an antigen. The pharmaceutical composition may comprise or consist of an adjuvant and Ku55933. The pharmaceutical composition may comprise or consist of an adjuvant, Ku55933, and an antigen. The methods may comprise or consist of administering an adjuvant and Tucidinostat. The methods may comprise or consist of administering an adjuvant, Tucidinostat, and an antigen. The pharmaceutical composition may comprise or consist of an adjuvant and Tucidinostat. The pharmaceutical composition may comprise or consist of an adjuvant, Tucidinostat, and an antigen. The methods may comprise or consist of administering an adjuvant and PD318088. The methods may comprise or consist of administering an adjuvant, PD318088, and an antigen. The pharmaceutical composition may comprise or consist of an adjuvant and PD318088. The pharmaceutical composition may comprise or consist of an adjuvant, PD318088, and an antigen. The methods may comprise or consist of administering an adjuvant and WNK463. The methods may comprise or consist of administering an adjuvant, WNK463, and an antigen. The pharmaceutical composition may comprise or consist of an adjuvant and WNK463. The pharmaceutical composition may comprise or consist of an adjuvant, WNK463, and an antigen. The methods may comprise or consist of administering an adjuvant and TRx0237 (LMTX) mesylate. The methods may comprise or consist of administering an adjuvant, TRx0237 (LMTX) mesylate, and an antigen. The pharmaceutical composition may comprise or consist of an adjuvant and TRx0237 (LMTX) mesylate. The pharmaceutical composition may comprise or consist of an adjuvant, TRx0237 (LMTX) mesylate, and an antigen.

The methods may comprise administering an adjuvant to a subject. The pharmaceutical compositions may comprise an adjuvant. The methods may comprise administering an additional adjuvant to the subject. The pharmaceutical composition may comprise an adjuvant that comprises or consists of one or more of 3′3′-cGAMP, Tri-DAP, MDP, and mIFN-B. The adjuvant may comprise or consist of a PRR agonist. The PRR agonist may be further defined as a TLR agonist. The TLR agonist may be an agonist to TLR1, TLR2/1, TLR2, TLR2/6, TLR3, TLR4, TLR5 TLR7, TLR8, TLR7/8, TLR9, and/or TLR11. Any of these agonists may be excluded in some aspects. The TLR agonist may comprise or consist of one or more of peptidoglycan, triacyl lipoproteins, lipoteichoic acid, peptidoglycan from Bacillus subtilis, peptidoglycan from E. coli 0111: B4, peptidoglycan from Escherichia coli K12, peptidoglycan from Staphylococcus aureus, atypical lipopolysaccharide (LPS), Leptospirosis LPS, Porphyromonas gingivalis LPS, a synthetic diacylated lipoprotein, FSL-1, Pam2CSK4, lipoarabinomannan from M. smegmatis, lipomannan from M. smegmatis; triacylated lipoproteins, Pam3CSK4, MALP-2 from mycoplasma, MALP-404 from mycoplasma, Borrelia burgdorferi OspA, Porin from Neisseria meningitidis, Porin from Haemophilus influenza, Propionibacterium acnes antigen mixtures, Yersinia LcrV, lipomannan from Mycobacterium, lipomannan from Mycobacterium tuberculosis, Trypanosoma cruzi GPI anchor, Schistosoma mansoni lysophosphatidylserine, Leishmania major lipophosphoglycan (LPG), Plasmodium falciparum glycophosphatidylinositol (GPI), zymosan, antigen mixtures from Aspergillus fumigatus, antigen mixtures from Candida albicans, antigen mixtures from measles hemagglutinin, double-stranded RNA, polyadenylic-polyuridylic acid (Poly(A:U)), polyinosine-polycytidylic acid (Poly(I:C)), polyinosine-polycytidylic acid high molecular weight (Poly(I:C) HMW), polyinosine-polycytidylic acid low molecular weight (Poly(I:C) LMW)), LPS from Escherichia coli, LPS from Salmonella species, monophosphoryl lipid A, flagellin, flagellin from B. subtilis, flagellin from P. aeruginosa, flagellin from S. typhimurium, single stranded RNAs with 6UUAU repeats, single stranded RNA homopolymer (ssPolyU naked), HIV-1 LTR-derived ssRNA (ssRNA40), ssRNA with 2 GUCCUUCAA repeats (ssRNA-DR)), imidazoquinoline compounds, imiquimod, Imiquimod VacciGrade™, Gardiquimod VacciGrade™, Gardiquimod™, adenine analog CL264, base analog CL307, guanosine analog loxoribine, TL8-506, thiazoquinoline compound CL075, imidazoquinoline compound CL097, 2Bxy, R848, R848 VacciGrade™, CpG ODN, and/or Toxoplasma gondii Profilin. In some aspects, any agonist may be excluded. The TLR agonist may also comprise a TLR agonist described herein.

The TLR agonist may be a TLR7/8 agonist. The TLR7/8 agonist may be a R848. The TLR agonist may be a TLR5 agonist. The TLR5 agonist may be flagellin. The TLR agonist may be a TLR9 agonist. The TLR9 agonist may be a CpG oligonucleotide. The CpG oligonucleotide may comprise or consist of CpG 1826.

In the methods of the disclosure, the one or more cytokines comprise one or more of IL-12p40, IP-1β, IL-1β, CCL4, TNF-α, and IFN-β (or any combination thereof). One or more cytokines of may IL-12p40, IP-10, IL-1β, CCL4, TNF-α, and IFN-β may be excluded.

The method may comprise or further comprise administering to the subject an additional cancer therapy. The additional cancer therapy may comprise chemotherapy, radiation therapy, immunotherapy, or a combination thereof. The additional cancer therapy may comprise immunotherapy. The subject may be a human subject. The subject may be a non-human primate, a laboratory animal, a mammal, a rat, dog, pig, horse, mouse, rabbit, goat, or cat. The subject may be one has not been diagnosed with cancer. The subject may be one that has been diagnosed with cancer. The subject may be one that was previously treated for cancer with therapy. The subject may have been in remission. The subject may be one that was determined to be resistant to the previous therapy. The pharmaceutical composition may be administered to the subject intratumorally.

A composition may be administered to a subject by intramucosal, intramuscular, parenteral, or subcutaneous administration. The adjuvant of (a), compound of (b) and/or antigen may be administered by intramucosal, intramuscular, parenteral, or subcutaneous administration. The adjuvant of (a), compound of (b) and/or antigen may be administered in the same composition or in separate compositions.

The method may be for preventing a disease in the subject. The method may be for treating a disease in a subject.

The methods and compositions may be combined with vaccine compositions known in the art, such as current flu vaccines, hepatitis B vaccines, or Covid vaccines. Specific examples include Fluzone and Heplisav, but others are also applicable. The vaccine composition may be provided in a separate composition or may be in the same composition. It may be provided before or after the composition of the disclosure or at substantially the same time.

A compound or composition as disclosed herein may be formulated for intramucosal, intramuscular, parenteral, or subcutaneous administration. The composition may further comprise a pharmaceutical excipient.

The preparation of the vaccine as the active immunogenic ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

Administration of vaccines according to the disclosure may be via any common route so long as the target tissue is available via that route in order to maximize the delivery of antigen to a site for maximum (or in some cases minimum) immune response. Administration will generally be by orthotopic, intradermal, mucosally, subcutaneous, intramuscular, intraperitoneal or intravenous injection. Other areas for delivery include: oral, nasal, buccal, rectal, vaginal or topical. Vaccines of the invention are preferably administered parenterally, by injection, for example, either subcutaneously or intramuscularly.

Vaccines may be administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, or in the range from about 10 mg to 50 mg. Suitable regimens for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of nucleic acid molecule or fusion polypeptides of this invention will depend, inter alia, upon the administration schedule, the unit dose of antigen administered, whether the vaccine composition is administered in combination with other therapeutic agents, and the immune status and health of the recipient.

A vaccine may be given in a single dose schedule or in a multiple dose schedule. A multiple dose schedule is one in which a primary course of vaccination may include, e.g., 1, 2, 3, 4, 5, 6, 7, 8, 9, or 10 separate doses, followed by other doses given at subsequent time intervals required to maintain and/or reinforce the immune response, for example, at 1, 2, 3, or 4 months for a second dose, and if needed, a subsequent dose(s) after several months. Periodic boosters at intervals of 1, 2, 3, 4, 5 years, such as 3 years, are desirable to maintain the desired levels of protective immunity.

A vaccine may be provided in one or more “unit doses”. Unit dose is defined as containing a predetermined-quantity of the vaccine calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. Unit dose of the present invention conveniently may be described in terms of mg/kg body weight. The dose of the NFκB inhibitor, adjuvant, or antigen may be at least, at most, or about 0.05, 0.10, 0.15, 0.20, 0.25, 0.5, 1, 10, 50, 100, 1,000 mg/kg, or any derivable range therein. Likewise the amount of vaccine delivered to an individual in vivo can vary from about 0.2 to about 8.0 mg/kg body weight. 0.1 mg/kg, 0.2 mg/kg, 0.3 mg/kg, 0.4 mg/kg, 0.5 mg/kg, 0.8 mg/kg, 1.0 mg/kg, 1.5 mg/kg, 2.0 mg/kg, 2.5 mg/kg, 3.0 mg/kg, 4.0 mg/kg, 5.0 mg/kg, 5.5 mg/kg, 6.0 mg/kg, 6.5 mg/kg, 7.0 mg/kg and 7.5 mg/kg (or any derivable range therein). The dosage of vaccine to be administered depends to a great extent on the weight and physical condition of the subject being treated as well as the route of administration and the frequency of treatment.

The methods of the disclosure may comprise administering one or more compositions two or more times. It is contemplated that the compositions may be administered 1, 2, 3, 4, 5, 6, 7,8,9, 10, 11, 12, 13 and/or 14 days apart and/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48,49, 50, 51 and/or 52 weeks apart and/or 1, 2, 3, 4, 5, 6, 7,8,9, 10, 11, 12, 13, 14, 15, 16, 17, 18, 19, 20, 21, 22, 23, 24, 36, 48, 60, 72, 84 or 96 months apart and/or 1, 2, 3, 4, 5, 6, 7, 8, 9, 10, 11, 12, 13, 14, 15, 16, 17, 18, 19 or 20 years apart (or any derivable range therein).

Also described is a kit comprising compositions of the disclosure and instructions for use.

Methods may further comprise testing the patient for an infection, such as a viral infection or diagnosing a patient with an infection, such as a viral infection.

Any embodiment discussed in the context of a composition may be implemented in any method embodiment discussed herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-1C: FIG. 1A Overview of how combinations of immunomodulators and PRRs produce antigen presentation and cytokines. FIG. 1B Examples of classes of modulators includes those specialists are specific towards one pattern recognition receptor, generalists that generally affect many PRRs. Antigen presentation and cytokines can be independently altered.

FIG. 1C Overview of informational screens to identify lead modulators. After the primary screen, modulators are down selected through PCA cutting, cytokines are measured for selected modulators and used to inform a quantitative vaccine score, lead candidates identified through the vaccine score are carried forward into preliminary in vivo studies, and two main classes of modulators, cytokine reducing modulators and antibody enhancing modulators, are identified.

FIGS. 2A-2B: Primary screen distributions-transcription factor activity. FIG. 2A Raw Dual measurements of transcription factor activity for IRF. FIG. 2B Raw Dual measurements of transcription factor activity for NF-κB. Measurements taken 24 hour after addition of modulator+agonist. Modulator (N=3147)+agonist (N=13) activity reported as a fold change compared to agonist alone activity. Inhibition and enhancement over multiple orders of magnitude is observed.

FIGS. 3A-3F: Primary screen down selection. FIG. 3A Modulators (N=3147) alone show little inherent activity in either NF-κB or IRF transcription factors. FIG. 3B Modulation of transcription factor for NF-κB with LPS shown to act independently of IRF transcription factor modulation FIG. 3C Modulators demonstrate different trends across agonists: general enhancers (upper circles), general inhibitors (lower circles), or specialist activity (middle circles) FIG. 3D Table summary of specialist modulators (active with one agonists) and generalist modulators (active with 12-13 agonists) FIG. 3E Principle component analysis with IRF and NF-κB transcription factor data from all modulators (N=3147) and agonists (N=8). FIG. 3F Compounds with minimal variability were removed by creating a circle centered on the origin with radius 1.75.

FIGS. 4A-4G: Secondary screen distributions—cytokine levels. THP-1 Cytokine distributions of 7 agonists Pam2 (FIG. 4A), Pam3 (FIG. 4B), Poly IC (FIG. 4C), LPS (FIG. 4D), Flagellin (FIG. 4E), R848 (FIG. 4F), and 3′3′-cGAMP (FIG. 4G) chosen for the secondary screen (N=720). Significant ability to modulate fold change (standardized to agonist alone controls) was observed for all agonists and cytokines. Values are normalized to agonist alone conditions. Nondetectable measurements of cytokines were given a value of 0.001 fold change for visual representation on a logarithmic graph.

FIGS. 5A-5E: Demonstration of modulator analysis—Vaccine Score. FIG. 5A Representative normalized cytokine distributions for one agonist, LPS (N=720). FIG. 5B Fold changes for cytokines were multiplied by weight factors and summed to obtain agonist specific “vaccine scores”. All agonist scores were combined to create the generalist score (N=720) FIG. 5C Cytokine production of a top vaccine score candidate, Bafetinib. FIG. 5D Cytokine production of a negative score candidate, MK-8353. FIG. 5E Cytokine production of a candidate for additional applications, TRx0237. Values indicate concentration normalized to agonist alone, values indicate fold change+SEM.

FIGS. 6A-6E: In vivo immunomodulation against OVA. FIG. 6A Schematic of in vivo study FIG. 6B Bafetinib serum anti-OVA IgG antibody level, day 49, n=4. Statistical analyses between agonist and agonist+Bafetinib were performed by an unpaired t test. FIG. 6C Modulators studied in vivo. FIG. 6D Systemic TNF-a levels 1 hour after vaccination with agonist, agonist+modulator, and vehicle (N=4) for CpG (TLR9). FIG. 6E Systemic TNF-a levels 1 hour after vaccination with agonist, agonist+modulator, and vehicle (N=4) for R848 (TLR7/8). Statistical analyses between agonist+modulator groups and agonist alone were performed by a one-way ANOVA test *P<0.05, **P<0.01, ***P<0.001.

FIGS. 7A-7B: High Throughput screening of NF-κB and IRF transcription factors. FIG. 7A Layout of Primary Screen plates (N=14 plates). FIG. 7B Schematic representation of the Primary Screen workflow.

FIGS. 8A-8C: Comparing NF-κB and IRF activity between similar agonists shows correlative activity. FIG. 8A MPLA (TLR4) vs LPS (TLR4) NF-κB transcription factor fold change. FIG. 8B MPLA vs LPS IRF transcription factor fold change. FIG. 8C Pam2 (TLR2/6) versus Pam3 (TLR2/1) NF-κB transcription factor fold change.

FIGS. 9A-9B: Viability Filter analysis identifies cytostatic and cytotoxic compounds in primary screen. FIG. 9A Using IncuCtye, two confluency masks were generated based of the imaged cell plates. FIG. 9B Non-viable modulators are identified. In total, 69.6% of modulators were viable in one or both viability masks.

FIGS. 10A-10B: Verification of PCA cutting analysis. FIG. 10A Full primary screen distribution (N=3147) for IRF transcription factor activity. FIG. 10B Distribution of Modulators chosen by PCA cutting analysis (N=720) shows retention of tails of NF-κB and IRF distributions and a significant reduction of modulators around a fold change of 1.

FIGS. 11A-11E: Cytokine screen workflow and optimization. FIG. 11A Schematic representation of the AlphaPlex workflow. FIG. 11B Table of beads for each cytokine duplex. FIG. 11C Correction factors for terbium and europium emission needed for multiplexing cytokines. FIG. 11D Representative standard curve used in interpolation. FIG. 11E Cytokines can be controlled independently within a particular agonist. For visual representation on a logarithmic graph, nondetectable measurements of cytokines were given a value of 0.0001-fold change.

FIGS. 12A-12B: Modulators by themselves do not stimulate cytokine production. FIG. 12A Representative data showing the addition of Modulator only+Agonist only samples (squares) produce minimal changes in secretion of cytokines, whereas Modulator+agonist samples (circles) yield significant modulation of IL-1β using Pam3 (TLR2/1). FIG. 12B Modulators alone produce minimal cytokine secretion (TNF-α shown). Addition of modulators to agonist modulate cytokine secretion over 3 orders of magnitude using Pam3.

FIGS. 13A-13B: Cytokine screen concentrations. FIG. 13A For the top active compounds, transcription factor activity correlated with an increase of decrease cytokine response. FIG. 13B IL-1β fold changes (N=720) of Pam2 versus Pam3.

FIG. 14: Comparison of 720 PCA selected compounds to 720 random subsection of primary screen. TNF-α comparison from a subsection of the primary screen (N=720) and the secondary library (N=720) show a significant increase in modulators that deviate from a fold change of one, showing evidence in the use of a PCA for narrowing compounds. ****P<0.0001 Statistical analysis performed was a F Test to compare variance. Kurtosis of log-transformed primary screen: 6.884, secondary screen: 5.907.

FIGS. 15A-15B: Vaccine score generation. FIG. 15A Table of weight factors for each cytokine for vaccine score. FIG. 15B Vaccine score for each agonist (N=720), individual agonist vaccine scores are summed up to create the generalist vaccine score.

FIGS. 16A-16B: In vivo immunomodulation with Bafetinib against OVA. FIG. 16A Bafetinib serum anti-OVA IgG antibody level, day 35, n=4. FIG. 16B Systemic TNF-α levels 1 hour after vaccination with agonist, agonist+modulator, and vehicle (N=4) for Flagellin (TLR5), R848 (TLR7/8) and CpG (TLR9). Bafetinib statistical analyses between agonist and agonist+Bafetinib were performed by an unpaired t test. *P<0.05, **P<0.01, ***P<0.001, *P<0.0001. n.s., not significant.

FIGS. 17A-17C: In vivo immunomodulation against OVA. FIG. 17A Systemic TNF-α levels 1 hour after vaccination with agonist, agonist+modulator, and vehicle (N=4) for Flagellin (TLR5). FIG. 17B Modulator serum anti-OVA IgG antibody level for R848 (TLR7/8) on day 35, n=4. FIG. 17C Modulator serum anti-OVA IgG antibody level for CPG (TLR9) on day 35, n=4. Statistical analyses between agonist and agonist+modulator were performed by an unpaired t test. *P<0.05, **P<0.01, ***P<0.001, ****P<0.0001. n.s., not significant. Modulator 1=Brivanib (BMS-540215); Modulator 2=Golvatinib; Modulator 3=KU-55933; Modulator 4=Selonsertib (GS-4997); Modulator 5=SL-327.

FIGS. 18A-18D: Preliminary in vivo immunomodulation against OVA formulated in liposomes. FIG. 18A Schematic of in vivo study. FIG. 18B Modulators studied in vivo were top performers in the constructed vaccine score. FIG. 18C Systemic TNF-α levels 1 hour after vaccination with agonist, agonist+Bafetinib, and vehicle (N=4) for R848 (TLR7/8) and CpG (TLR9). FIG. 18D Modulator serum anti-OVA IgG antibody level on day 49. Statistical analyses between agonist+modulator groups and agonist alone were performed by a one-way ANOVA test *P<0.05, **P<0.01, ***P<0.001, ****P<0.0001. n.s., not significant.

FIG. 19: High throughput screen distributions-Cell surface marker levels. THP-1Cytokine distributions of agonists (Pam2CSK4, Pam3CSK4, Poly I:C, LPS, Flagellin, R848, and 3′3′-cGAMP, only Pam3 shown) chosen for the secondary screen (N=720). Significant ability to modulate fold change (standardized to agonist alone controls) was observed for all agonists and cell surface markers. Values are normalized to agonist alone conditions.

FIGS. 20A-20D: In vivo immunomodulation against commercial vaccines. FIG. 20A Schematic of in vivo study, vaccinated mice IM against commercial vaccines (Fluzone, Heplisav and Typhim Vi, MMR, Shingrix (not pictured)). FIG. 20B Table of immunomodulators used. FIG. 20C Serum anti-OVA IgG antibody level, day 35, n=5. FIG. 20D Serum anti-OVA IgG antibody level, day 35, n=5. Statistical analyses between agonist and agonist+modulators were performed by an unpaired t test. Statistical analyses between agonist+modulator groups and agonist alone were performed by a one-way ANOVA test *P<0.05, **P<0.01, ***P<0.001, ****P<0.0001.

FIGS. 21A-21C: Viability filter analysis identifies cytostatic and cytotoxic compounds in primary screen. FIG. 21A Representative images of positive controls for IncuCyte analysis pathway Parameter Set 1. FIG. 21B Representative images of positive controls for IncuCyte analysis pathway Parameter Set 2. FIG. 21C Representative library plate viability. Wells/compounds shown in darkened boxes were determined as nonviable.

FIGS. 22A-22E: Preliminary in vivo immunomodulation against OVA formulated in liposomes. FIG. 22A Schematic of in vivo study. FIG. 22B Modulators studied in vivo were top performers in the constructed vaccine score. FIG. 22C Systemic IFN-B levels 1 hour after vaccination with vehicle, R848, and agonist. FIG. 22D Systemic IL-6 levels 1 hour after vaccination with vehicle, R848, and agonist. FIG. 22E Systemic TNF-α levels 1 hour after vaccination with vehicle, R848, and agonist.

FIGS. 23A-23B: Immunomodulators Suppress the Inflammatory Immune Responses of mRNA Vaccine. FIG. 23A PME-564 and PME-2834 reduce supernatant IL-6 cytokine production measured 24 hours after addition of immunomodulator in BMDCs (n=3) treated. FIG. 23B PME-564 and PME-2834 reduce supernatant TNF-α cytokine production measured 24 hours after addition of immunomodulator in BMDCs (n=3) treated with LNPs.

FIGS. 24A-24B: Immunomodulators Suppress the Inflammatory Immune Responses of mRNA Vaccine. FIG. 24A Intracellular IL-6 cytokines of BMDCs measured 48 hours after addition of immunomodulator and LNP (n=3) show reduced cytokines in combination with modulators. FIG. 24B Intracellular TNF-α cytokines of BMDCs measured 48 hours after addition of immunomodulator and LNP (n=3) show reduced cytokines in combination with modulators.

FIGS. 25A-25C: Preliminary in vivo study shows reduction in cytokines, antibody levels maintained. C57BL/6J (n=4) were immunized intramuscularly with home-made SARS-CoV-2 mRNA vaccine (WA-1/Omicron, 5 ug/dose) and immunomodulators (1.5 μmol). FIG. 25A Preliminary in vivo IL-6 cytokine measured 1 hr post injection. LNP+modulators lowered systemic cytokines. FIG. 25B Antibody titers measured 7d post injection. No difference between ‘mRNA vaccine alone’ and either modulator group. FIG. 25C Antibody titers measured 28d post injection. Statistical analyses between agonist+modulator groups and agonist alone were performed by ANOVA *P<0.05, **P<0.01, ***P<0.001, ****P<0.0001.

### DESCRIPTION OF ILLUSTRATIVE EMBODIMENTS

Imidazoquinolinone derivatives that activate Toll like receptor (TLR) 7/8 are small molecule immune potentiators (SMIPs) that have potent activity as vaccine adjuvants and as anti-tumor agents. However, these molecules have high bioavailability that results in unacceptable levels of systemic inflammation due to adjuvant toxicity greatly limiting their use.

Small molecule NF-κB inhibitors can be used to potentiate cytidine phosphate guanosine (CPG, a TLR 9 agonist) in vaccine formulations. The small molecule NF-κB inhibitors capsaicin and honokiol were shown to reduce pro-inflammatory systemic IL-6 and TNF-α levels while maintaining vaccine protective effects.¹⁹ However, this effect was not observed when the in-vivo experiments were repeated with R848 as an adjuvant. This is likely due to the high diffusion of the small molecule adjuvant and the immune potentiators.

The present disclosure is based, at least in part, on the design of hybrid molecules in which an imidazoquinolinone derivative²⁰ was covalently linked through an conjugatable amine handle to vanilloid, catechol and honokiol¹⁹ derivatives in order to reduce the degree of diffusion. Using in vitro assays, a mini library of synthesized dimers was screened and viable candidates were selected for further in vivo experiments. Mice were vaccinated with ovalbumin as a model antigen treated with the synthesized dimers. The results demonstrated that these dimers reduce systemic toxicity to baseline levels while maintaining the adjuvanticity in a vaccine formulation. Additionally, select dimers increased survivability in a CT26 WT mouse colon carcinoma tumor model while eliciting low adjuvant toxicity.

### I. Definitions

The term “adjuvant” as used herein refers to substances, which when administered prior, together or after administration of an antigen, accelerate, prolong and/or enhance the quality and/or strength of an immune response to the antigen in comparison to the administration of the antigen alone.

As used herein, the term “vaccine” describes a composition which can be administered to humans or to animals in order to induce an immune system response; this immune system response can result in a production of antibodies or simply in the activation of certain cells, for example antigen-presenting cells, T lymphocytes and/or B lymphocytes. The vaccine may be capable of producing an immune response that leads to the production of neutralizing antibodies in the patient with respect to the antigen provided in the vaccine. The vaccine can be a composition for prophylactic purposes or for therapeutic purposes, or both.

As used herein, the term “antigen” refers to any antigen that can be used in a vaccine, whether it involves a whole microorganism or a portion thereof, and various types: (e.g., peptide, protein, glycoprotein, polysaccharide, glycolipid, lipopeptide, etc). Thus, the term “antigen” refers to a molecule that can initiate a humoral and/or cellular immune response in a recipient of the antigen. The antigen may be a molecule that causes a disease for which a vaccination would be advantageous treatment. The antigen may comprise a substance used to stimulate the production of antibodies and provide immunity against one or several diseases, prepared from the causative agent of a disease, its products, or a synthetic substitute, treated to act as an antigen without inducing the disease. The antigen may comprise a peptide or polypeptide.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, and pH buffering agents.

As used herein, the term “agonist” refers to a molecule that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another molecule so that the other molecule directly binds to the receptor. An agonist may be referred to as an agonist of a particular receptor or family of receptors (e.g., a TLR agonist).

“Individual, “subject,” and “patient” are used interchangeably and can refer to a human or non-human.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term consisting essentially of may include the listed active ingredients, such as the recited dimer, and also any unrecited buffers, pharmaceutical excipients, etc., but exclude any other active ingredients, such as other hybrid molecules.

### Antigens

The term “antigen” as used herein refers to a molecule against which a subject can initiate a humoral and/or cellular immune response. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. In certain compositions and methods of the disclosure, the antigen is a peptide.

The inventors have demonstrated that the hybrid molecules disclosed herein reduce systemic toxicity to baseline levels while maintaining the adjuvanticity in a vaccine formulation. Antigens useful in methods and compositions of the disclosure include, for example, antigenic components from Anthrax, Cancer, Chikungunya, Dengue (1,2,3,4-Dengue Fever), Diphtheria, E. coli, Shiga toxin-producing (STEC), Ebola, Non-Polio Enterovirus, Enterovirus D68 (EV-D68), Gonorrhea, Hepatitis A (Hep A), Hepatitis B (Hep B), Hepatitis C (Hep C), Hepatitis D (Hep D), Hepatitis E (Hep E), Herpes, Shingles, HIV, HPV, Influenza, Malaria, Measles, Viral Meningitis, Bacterial Menigitis, Mumps, Norovirus, Pertussis, Plague; Bubonic, Septicemic, Pneumonic, Pneumococcal Disease, Poliomyelitis (Polio), Pustular Rash diseases (Small pox, monkeypox, cowpox), Q-Fever, Rabies, Salmonellosis gastroenteritis (Salmonella), Severe Acute Respiratory Syndrome, Shigellosis gastroenteritis (Shigella), Smallpox, Tetanus, Tuberculosis, Varicella (Chickenpox), Viral Hemorrhagic Fever (Ebola, Lassa, Marburg), West Nile Virus, Yellow Fever, Yersenia(Yersinia), and Zika Virus Infection. It is contemplated that one or more of the antigens and antigenic components listed in this paragraph can be specifically excluded.

Further examples of antigens useful in the methods and compositions of the disclosure are provided below and throughout the disclosure.

### A. Viral Antigens

Examples of viral antigens include, but are not limited to, retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B. and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, e's. Fields, B. N. and Knipe, D. M. (Raven Press, New York, 1991) for additional examples of viral antigens. It is contemplated that one or more of the antigens and antigenic components listed in this paragraph can be specifically excluded.

### B. Bacterial Antigens

Bacterial antigens which can be used in the compositions and methods of the disclosure include, but are not limited to, pertussis bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component. Also included with the bacterial antigens described herein are any other bacterial, mycobacterial, mycoplasmal, rickettsial, or chlamydial antigens. It is contemplated that one or more of the antigens and antigenic components listed in this paragraph can be specifically excluded in methods and compositions of the disclosure.

### C. Fungal Antigens

Fungal antigens which can be used in the compositions and methods of the disclosure include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. It is contemplated that one or more of the antigens and antigenic components listed in this paragraph can be specifically excluded in methods and compositions of the disclosure.

### D. Parasite Antigens

Examples of protozoa and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components. It is contemplated that one or more of the antigens and antigenic components listed in this paragraph can be specifically excluded in methods and compositions of the disclosure.

### E. Tumor Antigens

Tumor antigens which can be used in the compositions and methods of the disclosure include, but are not limited to, telomerase components; multidrug resistance proteins such as P-glycoprotein; MAGE-1, alpha fetoprotein, carcinoembryonic antigen, mutant p53, immunoglobulins of B-cell derived malignancies, fusion polypeptides expressed from genes that have been juxtaposed by chromosomal translocations, human chorionic gonadotropin, calcitonin, tyrosinase, papillomavirus antigens, gangliosides or other carbohydrate-containing components of melanoma or other tumor cells. It is contemplated by the disclosure that antigens from any type of tumor cell can be used in the compositions and methods described herein. It is contemplated that one or more of the antigens and antigenic components listed in this paragraph can be specifically excluded in methods and compositions of the disclosure.

### F. Antigens Relating to Autoimmunity

Antigens involved in autoimmune diseases, allergy, and graft rejection can be used in the compositions and methods of the disclosure. For example, an antigen involved in any one or more of the following autoimmune diseases or disorders can be used in the present disclosure: diabetes mellitus, arthritis (including rheumatoid arthritis, juvenile rheumatoid arthritis, osteoarthritis, psoriatic arthritis), multiple sclerosis, myasthenia gravis, systemic lupus erythematosus, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. An antigen can also be an altered peptide ligand useful in treating an autoimmune disease. It is contemplated that one or more of the antigens and antigenic components listed in this paragraph are specifically excluded in methods and compositions of the disclosure. It is further contemplated that autoantigens can be specifically excluded in methods and compositions of the disclosure.

Examples of miscellaneous antigens which can be used in the compositions and methods of the disclosure include endogenous hormones such as luteinizing hormone, follicular stimulating hormone, testosterone, growth hormone, prolactin, and other hormones, drugs of addiction such as cocaine and heroin, and idiotypic fragments of antigen receptors such as Fab-containing portions of an anti-leptin receptor antibody.

### II. Adjuvants

Aspects of the present disclosure include adjuvants and methods for administering adjuvants to a subject. The immunogenicity of a particular composition can be enhanced by the use of non-specific stimulators of the immune response, known as adjuvants. “Adjuvant” as used herein refers to a substance, which when administered prior, together or after administration of an antigen, accelerates, prolongs and/or enhances the quality and/or strength of an immune response to the antigen in comparison to the administration of the antigen alone. Adjuvants that may be used in accordance with aspects include, but are not limited to, IL-1, IL-2, IL-4, IL-7, IL-12, γ-interferon, GM-CSF, BCG, aluminum hydroxide, MDP compounds, such as thur-MDP and nor-MDP, CGP (MTP-PE), lipid A, and monophosphoryl lipid A (MPL). Other example adjuvants may include complete Freund's adjuvant (a non-specific stimulator of the immune response containing killed Mycobacterium tuberculosis), incomplete Freund's adjuvants, and/or aluminum hydroxide adjuvant.

In some aspects, an adjuvant of the disclosure is a TLR agonist or a pattern recognition receptor (PRR) agonist. Use of TLR agonists as adjuvants is described in, for example, Li et al., TLR Agonists as Adjuvants for Cancer Vaccines. Adv Exp Med Biol. 2017;1024:195-212 (incorporated herein by reference in its entirety). A PRR agonist describes any molecule that, directly or indirectly, activates a PRR or stimulates PRR signaling. PRRs include cell surface receptors (e.g., toll-like receptor (TLR) agonists) and intracellular receptors (e.g., RIG-I-like receptors). Examples of PRRs which may be targeted by agonists of the present disclosure include NOD-like receptors, RIG-I-like receptors, STING receptors, and toll-like receptors. In some embodiments, disclosed herein are PRR agonists, wherein a PRR agonist is a NOD-like receptor agonist, a RIG-I-like receptor agonist, a STING agonist, or a TLR agonist. In some embodiments, a PRR agonist of the present disclosure is a TLR agonist.

Aspects of the present disclosure relate to TLR agonists, including polymers comprising a TLR agonist or derivative thereof. A TLR agonist may be any molecule that, directly or indirectly, activates a TLR and/or stimulates TLR signaling. In some cases, a TLR agonist is a molecule that binds directly to a TLR. In some aspects, disclosed are immunomodulators comprising one or more TLR agonists linked by a polypeptide backbone.

In some embodiments, the TLR agonist is one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111: B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CL097, 2Bxy, R848, or R848 VacciGrade™), TLR9 (e.g., CpG ODNs); and TLR11 (e.g., Toxoplasma gondii Profilin). In some embodiments, the TLR agonist is an amphiphilic TLR agonist. In some embodiments, the TLR agonist is a TLR 2/6 agonist, for example Pam2CSK4 or Pam3CSK4. In some embodiments, the TLR agonist is a hydrophobic TLR agonist. In some embodiments, the TLR agonist is a TLR 7, TLR 8, or TLR 7/8 agonist, for example 2Bxy or imidazoquinoline. In some aspects, a TLR agonist of the disclosure is imidazoquinoline. In certain embodiments, the TLR agonist is a specific agonist listed above. Derivatives of any of the TLR agonists listed above are also contemplated herein, and TLR agonists of the disclosure encompass any derivative of the molecules listed above having TLR agonist activity. It is further contemplated that any TLR agonist disclosed herein can be specifically excluded in methods and compositions of the disclosure.

In further embodiments, the TLR agonist is one that agonizes either one TLR or two TLRs specifically. In some embodiments, linked TLR agonists comprise different types of TLR agonists (e.g., TLR agonists capable of activating different classes of TLRs). Alternatively, linked TLR agonists may comprise the same type of TLR agonist.

In some embodiments, disclosed herein are small molecule compounds suitable for use as TLR agonists. Examples of small molecule TLR agonists include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-substituted tetrahydroimidazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

In certain embodiments, the TLR agonist is an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the TLR agonist is a sulfonamide-substituted imidazoquinoline amine. In alternative embodiments, the TLR agonist can be a urea-substituted imidazoquinoline ether. In another alternative embodiment, the TLR agonist can be an aminoalkyl-substituted imidazoquinoline amine. In one particular embodiment, the TLR agonist is 4-amino-α,α,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-y1]ethoxy}ethyl)-N-methylmorpholine-4-carboxamide. In another alternative embodiment, the TLR agonist is 1-(2-amino-2-methylpropyl)-2-(ethoxymethyl)-1H-imidazo[4,5-c]quinolin-4-amine. In another alternative embodiment, the TLR agonist is N-[4-(4-an-no-2-ethyl-1H-imidazo[4,5-c]quinolin-1-yl)butyl]methanesulfonamide. In yet another alternative embodiment, the TLR agonist is N-[4-(4-amino-2-propyl-1H-imidazo[4,5-c]quinolin-1-yl)butyl]methanesulfonamide.

In certain embodiments, the TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines.

### III. Pharmaceutical Compositions

Administration of the compositions will typically be via any common route. This includes, but is not limited to parenteral, orthotopic, intradermal, subcutaneous, intramuscular, intraperitoneal, intranasal, or intravenous injection. A vaccine composition may be inhaled (e.g., U.S. Pat. No. 6,651,655, which is specifically incorporated by reference). Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, for example about 25% to about 70%.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of an antibody are applicable. These are believed to include oral application on a solid physiologically acceptable base or in a physiologically acceptable dispersion, parenterally, by injection and the like. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2 day to twelve week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The hybrid molecules can be formulated for parenteral administration, e.g., formulated for injection via the intravenous, intradermal, intramuscular, sub-cutaneous, or even intraperitoneal routes. The composition may be administered by intradermal injection. The composition may be administered by intravenous injection. The composition may be administered by intramuscular injection. Compositions of the disclosure can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### IV. Methods of Treatment

As discussed above, the compositions and methods of using these compositions can treat a subject (e.g., prevent an infection, evoke a robust immune response to an antigen, or reduce or prevent tumor proliferation) having, suspected of having, having one or more symptoms of, or at risk of developing an infection, cancer, or related disease.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the invention in a recipient patient. Treatment or therapy can be an active immune response induced by administration of immunogen or a passive therapy effected by administration of antibody, antibody containing material, or primed T-cells.

The presence of a cell-mediated immunological response can be determined by proliferation assays (CD4(+)T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

The methods may include treatment for or prevention of a disease or condition caused by a pathogen. Furthermore, in some examples, treatment comprises administration of other agents commonly used against viral infection, such as one or more antiviral or antiretroviral compounds.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

A subject may be administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 micrograms, mg, ug/kg, or mg/kg (or any range derivable therein), of hybrid molecule or composition.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. The patient may be administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms of an infection have disappeared or been reduced.

### V. Combination Therapy

The compositions and related methods, particularly administration of a composition comprising a composition of the disclosure, may also be used in combination with the administration of one or more additional therapies.

A therapy may be used in conjunction with antiviral or anti-retroviral treatment. A therapy may be used in conjunction with an anti-cancer treatment (e.g., chemotherapeutic, cancer immunotherapeutic, etc.). The therapy may precede or follow the other agent treatment by intervals ranging from minutes to weeks. Where the other agents and/or proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other, for example within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

A vaccine may be administered as part of a prime/boost strategy. A priming vaccine dose can be administered in any of the methods described herein. A vaccine boost can be administered through the use of a second vaccine, either of the same type or from a different type of vaccine. Examples of such different vaccines include naked DNA vaccines or a recombinant poxvirus.

Various combinations of therapy may be employed, for example adjuvant is “A” and NFkB inhibitor is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions to a patient/subject will follow general protocols for the administration of such compounds, taking into account the toxicity, if any, of the composition. It is expected that the treatment cycles would be repeated as necessary. It is also contemplated that various standard therapies, such as hydration, may be applied in combination with the

### VI. Cancer Therapy

The disclosed methods may comprise administering a cancer therapy to a subject or patient. The cancer therapy may comprise a local cancer therapy. The cancer therapy may exclude a systemic cancer therapy. The cancer therapy may exclude a local therapy. The cancer therapy may comprise a local cancer therapy without the administration of a system cancer therapy. The cancer therapy may comprise administering a dimer of the present disclosure. The cancer therapy may comprise a radiotherapy. The cancer therapy may comprise a chemotherapy. The cancer therapy may comprise an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. The cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. The cancer may be a Stage I cancer. The cancer may be a Stage II cancer. The cancer may be a Stage III cancer. The cancer may be a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Disclosed are methods for treating cancer originating from the colon. The cancer may be colon cancer. The cancer may be colorectal cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

Radiotherapy, such as ionizing radiation, may be administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

The radiotherapy can comprise external radiotherapy, internal radiotherapy, radioimmunotherapy, or intraoperative radiation therapy (IORT). The external radiotherapy may comprise three-dimensional conformal radiation therapy (3D-CRT), intensity modulated radiation therapy (IMRT), proton beam therapy, image-guided radiation therapy (IGRT), or stereotactic radiation therapy. The internal radiotherapy may comprise interstitial brachytherapy, intracavitary brachytherapy, or intraluminal radiation therapy. The radiotherapy may be administered to a primary tumor.

The amount of ionizing radiation is greater than 20 Gy and may be administered in one dose. The amount of ionizing radiation may be 18 Gy and is administered in three doses. The amount of ionizing radiation may be at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of radiotherapy administered to a subject may be presented as a total dose of radiotherapy, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of radiation may be at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### B. Cancer Immunotherapy

The methods may comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

The methods and compositions of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. The inhibitor may comprise the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

The inhibitor may comprise the heavy and light chain CDRs or VRs of tremelimumab or ipilimumab. The inhibitor may comprise the CDR1, CDR2, and CDR3domains of the V_{H} region of tremelimumab or ipilimumab, and the CDR1, CDR2 and CDR3 domains of the V_{L} region of tremelimumab or ipilimumab. The antibody may be one that competes for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3′s main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

The immune checkpoint inhibitor may be an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

The inhibitor may comprise the heavy and light chain CDRs or VRs of an anti-LAG3 antibody. The inhibitor may comprise the CDR1, CDR2, and CDR3 domains of the V_{H} region of an anti-LAG3 antibody, and the CDR1, CDR2 and CDR3 domains of the V_{L} region of an anti-LAG3 antibody. The antibody may have at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

The immune checkpoint inhibitor may be an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

The inhibitor may comprise the heavy and light chain CDRs or VRs of an anti-TIM-3 antibody. The inhibitor may comprise the CDR1, CDR2, and CDR3 domains of the V_{H} region of an anti-TIM-3 antibody, and the CDR1, CDR2 and CDR3 domains of the V_{L} region of an anti-TIM-3 antibody. The antibody may have at least about 70, 75, 80, 85, 90, 95, 97, or 99% (or any derivable range or value therein) variable region amino acid sequence identity with the above-mentioned antibodies.

### 2. Activation of Co-Stimulatory Molecules

The immunotherapy may comprise an activator of a co-stimulatory molecule. The activator may comprise an agonist of B7-1 (CD80), B7-2 (CD86), CD28, ICOS, OX40(TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Methods and compositions of the disclosure include patients that have been previously treated for a therapy described herein, are currently being treated for a therapy described herein, or have not been treated for a therapy described herein. The patient may be one that has been determined to be resistant to a therapy described herein. The patient may be one that has been determined to be sensitive to a therapy described herein. For example, the patient may be one that has been determined to be sensitive to an immune checkpoint inhibitor therapy based on a determination that the patient has or previously had pancreatitis.

### C. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be used as a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### E. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### F. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—High Throughput Screening of Immunomodulators for Vaccine Adjuvants

### A. Materials and Methods

### 1. Raw Dual Cell Culture

RAW-Dual cells were purchased from InvivoGen and were cultured with DMEM and 10% FBS/1% P/S.

### 2. NF-κB and IRF Transcription Factor Screening

RAW-Dual cells (InvivoGen) were plated at 50,000 cells per well in 45 μL of DMEM with 5% HI FBS from col 2-23 in clear 384 well plates. Cells attached at room temperature for 1 hour. 50 nL of 10 mM modulator libraries were added by Janus G3 via pintool to experimental wells (cols 3-22) for a final concentration of 10 μM. Following 1 hour incubation, 5 μL of PRR agonist was added via a MultiDrop Combi liquid handler (col 3-23). Cells were incubated at 37 C and 5% CO₂ overnight. 20 hours later, 12.5 μL of QuantiLuc Plus was plated in an opaque, white 384 well plate. 5 μL of cell supernatant was then added before measuring luminescent values on a BioTek Synergy NEO2 plate reader. In parallel, 15 μL of 5× concentrated QuantiBlue was added directly to the remaining cell supernatant. Absorbance values were measured at varying time intervals at 620 nm.

### 3. Viability Monitoring

Viability was monitored after overnight modulator addition by monitoring confluency via IncuCyte imaging. Two sets of parameters were used to create a confluency masks over all imaged wells. Modulators were determined toxic if both sets of confluency masks were <70% of those of resting cells. Confluency masks were quantified using IncuCyte software. This methodology was validated with select library plates using a traditional CellTiter Glo assay (Promega).

### 4. THP-1 Cell Culture

THP-1 cells were purchased from ATCC and were cultured in RPMI 1640 with 10% Biotin free HI-FBS and 1% P/S. Cells were maintained between 0.2-1.0×10⁶ cells/mL.

### 5. Cytokine Level Screening

THP-1 cells were seeded at 50,000 cells per well in 45 μL of biotin-free RPMI+5% HI-FBS in clear 384 well plates. Following 24h incubation, 50 nL of 10 mM modulator libraries were added by Janus G3 via pintool to experimental wells (cols 3-22) for a final concentration of 10 μM. After 1 hour, 5 μL of agonist was added via a MultiDrop Combi liquid handler (col 3-23). The following day, 5 μL of supernatant was transferred to white low volume ProxiPlates. 10 μL of a prepared acceptor bead (10 μg/mL final conc), biotinylated antibody (1 nM final conc) mixture was added via liquid handler to the cell supernatant. After 1 hr incubation at RT, 5 μL of donor beads (40 μg/mL final conc) were added in the dark. After an additional 1 hr incubation, plates were read on a plate reader with AlphaPlex filters for europium (615 nm) and terbium (545 nm) emission. A separate plate was run each day containing a standard curve of known analyte for interpolation purposes.

6. In Vivo studies
a. Animals

All animal procedures were performed under a protocol approved by the University of Chicago Institutional Animal Care and Use Committee (IACUC). 6-to-8-week-old C57/B6 female mice were purchased from the Jackson laboratory. All vaccinations were administered intramuscularly in the hind leg. Blood was collected from the submandibular vein at time points indicated.

VacciGrade Ovalbumin and AddaVax was purchased from InvivoGen. VacciGrade CpG ODN 1826, ultrapure flagellin, and VacciGradeR848 were purchased from InvivoGen. Modulators were purchased through Selleck Chemicals.

b. Vaccinations

Mice were lightly anesthetized with isoflurane and injected intramuscularly in the hind leg with an injection volume of 50 μL containing antigen, adjuvant, and a DMSO/AddaVax combination. Antigen dose was as follows: OVA (100 μg)). Agonists doses were as follows: Flagellin, 10 μg; R848, 50 μg; CpG, 50 μg. Modulators were added at 1.5 μmol.

c. Plasma Cytokine Analysis

Blood was collected from mice at specified time points in 0.2 mL of heparin-coated collection tubes (VWR Scientific). Plasma was isolated via centrifugation 1500×g at 4 C for 15 min. Samples were collected and stored at −80° C. until use. Plasma was analyzed using Bio-Legend's LegendPlex™ Mouse Inflammation Cytokine Panel (13-Plex) according to the manufacturer's protocol. Samples were analyzed using an ACEA NovoCyte Flow Cytometer. Data were analyzed using LEGENDplex™ Data Analysis Software Suite and GraphPad Prism.

d. Antibody Quantification

Mice were vaccinated with indicated formulations. Blood was collected at time points indicated in 0.2 mL heparin-coated collection tubes (VWR Scientific). Plasma was isolated via centrifugation 1500×g at 4 C for 15 min. Samples were collected and stored at −80° C. until use. Samples were analyzed using an anti-OVA IgG ELISA kit (Chondrex) according to the specified protocol. Antibody levels were analyzed using a Multiskan FC plate reader (Thermo Fisher) and absorbance was measured at 450 nm. Data were analyzed using GraphPad Prism.

### 7. Statistics and Replicates

Data are plotted and reported in the text as the means±SEM. Sample size is as indicated in biological replicates in all in vivo and in vitro experiments. The sample sizes were chosen based on preliminary experiments or literature precedent indicating that the number would be sufficient to detect significant differences in mean values should they exist. P values were calculated using a one-way analysis of variance (ANOVA) and Tukey post hoc test or two-tailed unpaired heteroscedastic t test where appropriate. All experiments have been repeated (sometimes with minor variations because of reagents and materials), and replication was successful.

### B. Results

### 1. Primary Screening Demonstrates Control of NF-κB and IRF Transcription Factor Activity

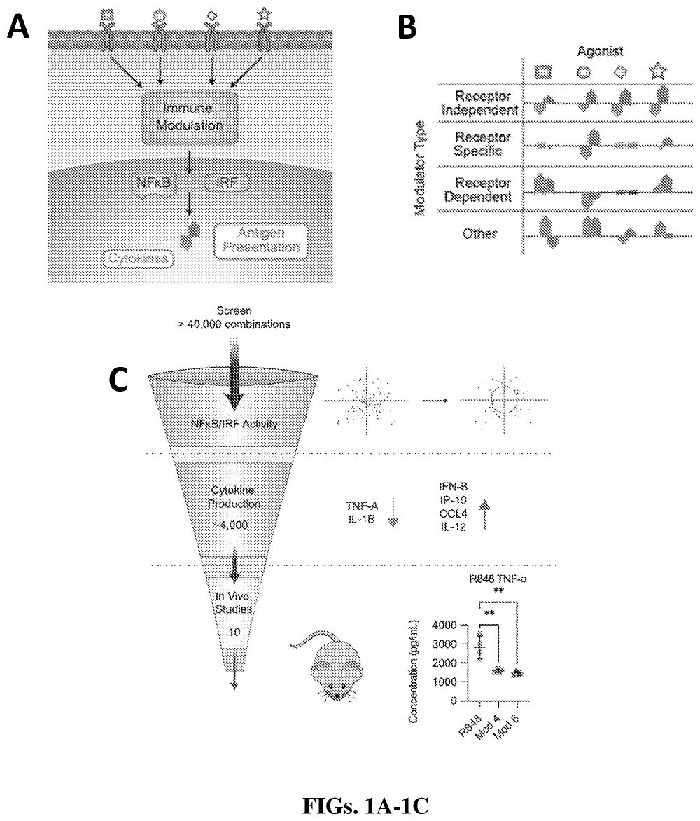

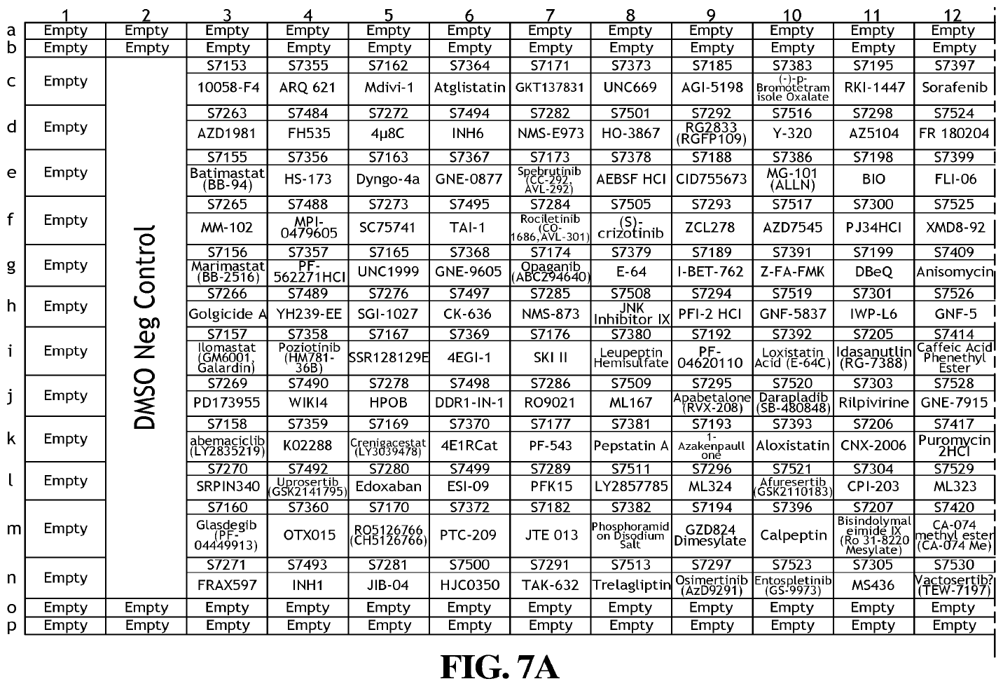

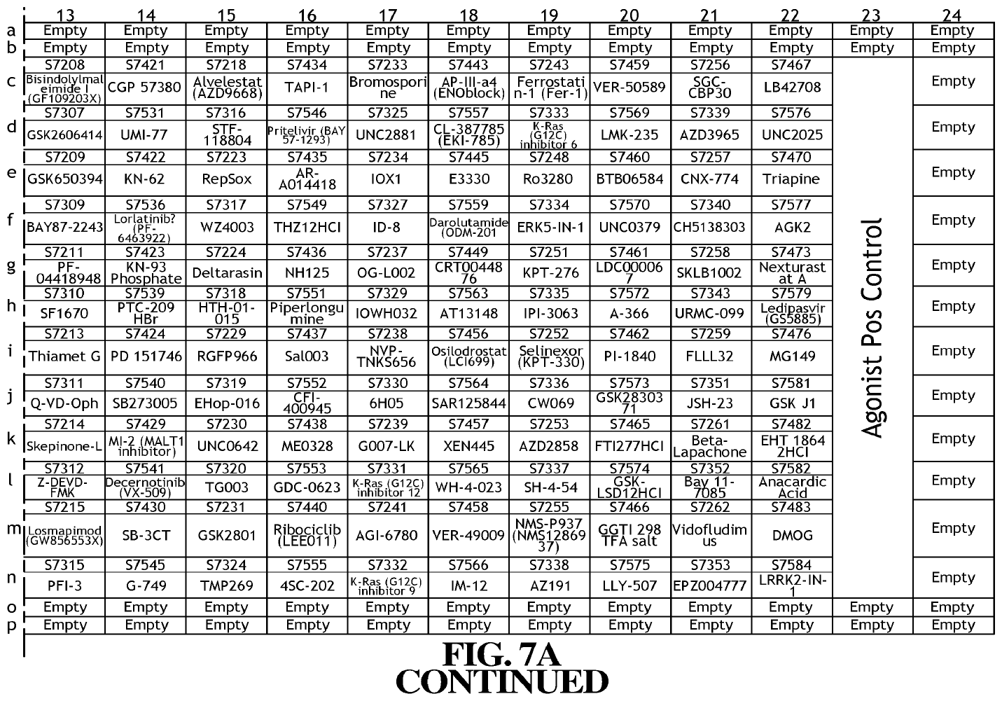

To identify new adjuvants, the inventors conducted a high throughput screen (FIG. 1C) to examine differing levels of innate immune cell NF-κB and IRF activity after treatment with immunomodulators in combination with PRR agonists. The inventors chose RAW-Dual macrophages to achieve this goal—a cell line which quantitatively reports NF-κB and IRF activity via secreted alkaline phosphatase (SEAP) and Lucia luciferase under control of the respective transcription factor promoters (11, 12). For the initial screen, the inventors explored a targeted library of small molecules: 246 NF-κB and IRF inhibitors and 2,895 pathway specific inhibitors (Table 1, FIGS. 7A-7B). Many of the included compounds had been previous studied, some even receiving FDA approval for other therapeutic applications. The inventors hypothesized this library had an increased likelihood of modulating the desired immune signaling pathways. The inventors observed this library's modulation of 14 PRR agonists, with a heavy emphasis on toll-like receptors (TLRs) (Table 1) (13). The inventors included this wide range of agonists to better understand trends in modulator activity across similar or distinct PRRs and signaling pathways.

TABLE 1
Table of compounds that compose the primary screen

| Library | Source | Number |
|---|---|---|
| Inhibitors | Selleckchem | 2,790 |
| NF-κB | Selleckchem | 41 |
| NF-κB | MedChem Express | 206 |
| Tolerance | Various | 104 |
| | Total | 3,141 |

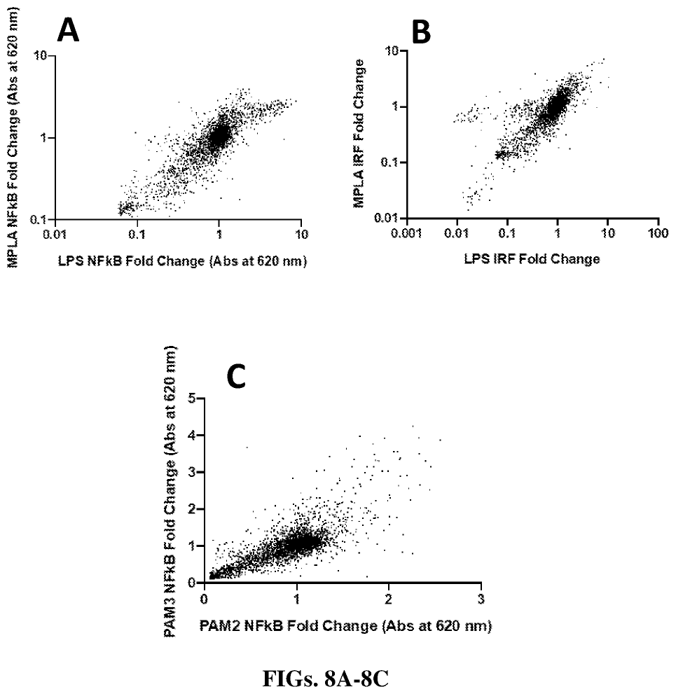

To screen this initial library for activity in modulating NF-κB and IRF activity, the inventors seeded 50,000 cells in 384 well plates in 45 μL of complete media. The inventors transferred immunomodulator compounds from source plates to a final concentration of 10 μM. Following 1 hr incubation at 37° C., one of fourteen PRR agonists was added in 5 μL of media to achieve the desired concentration (Table 2). Cells were incubated with agonist overnight until transcription factor activity was analyzed. This activity was measured via use of the proprietary substrates QUANTI-Blue and QUANTI-Luc for absorbance and luminescence readings. With a supernatant based assay, the inventors observed both NF-κB and IRF activity simultaneously from the same well. To ensure consistent and quality results, the inventors optimized this screening workflow, from biological aspects including cell seeding density, incubation time, agonist concentration, to assay intricacies such as liquid handling, reagent volume, and plate uniformity (FIGS. 8A-8C) (14, 15).

TABLE 2
Agonists used in primary screen, agonist targets,
pathways activated, and working concentrations

| Agonist | Target | NFκB | IRF | Conc. (ug/mL) |
|---|---|---|---|---|
| Pam2CSK4 | TLR2/1 | X | | 0.1 |
| Pam2CSK4 | TLR2/6 | X | | 0.1 |
| Poly I:C | TLR3 | | X | 1 |
| MPLA | TLR4 | X | X | 0.25 |
| LPS | TLR4 | X | X | 0.1 |
| Flagellin | TLR5 | X | X | 1 |
| Imiquimod | TLR7 | X | X | 2.5 |
| TL8-506 | TLR8 | | X | 0.1 |
| ODN 1826 | TLR9 | X | X | 0.5 |
| 3′3′-cGAMP | STING | | X | 10 |
| Tri-DAP | NOD1 | X | | 25 |
| MDP | NOD2 | X | | 10 |
| mIFN-β | IFNAR1/2 | X | X | 0.00833 |

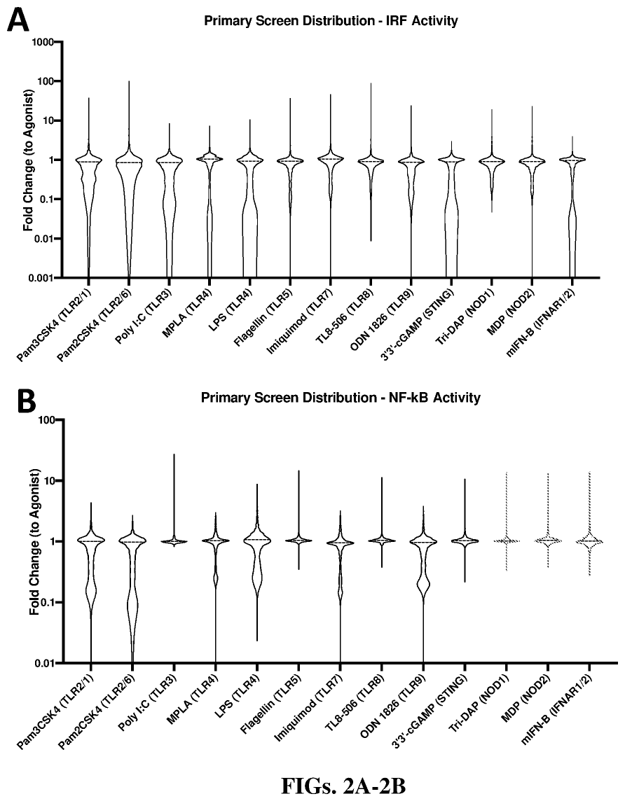

The inventors' initial screening approach presented a unique challenge regarding this assay optimization and analysis. Most high throughput screens seek to either maximize or minimize a desired output (16-18). For example, screening for a novel TLR4 agonist might seek to boost activity above resting levels. In this case, however, the inventors modulated existing PRR activity, and thus compares the modulator+agonist combination to agonist alone, reporting the modulation as a fold change. While the inventors anticipated finding inhibition of both immune pathways, the inventors were surprised to see enhancement of transcription factor activity. In fact, modulators enhanced or inhibited NF-κB and IRF over 100+ fold change while maintaining cell viability (FIGS. 2A-2B). For all 14 agonists studied, modulators produced significant enhancement or inhibition. This modulation persisted even when using potent agonists with a base-level high levels of activity. For example, modulation of 3′3′-cGAMP, a STING agonist, showed a fivefold increase in IRF activity-a result which was surprising as very few molecular entities have achieved higher activation of STING than 3′3′-cGAMP (19). Perhaps the most surprising, LPS, a TLR4 agonist often considered the standard of potent activity, showed a tenfold increase in NF-κB activity. This result provided a challenge as a screen that generates both inhibition and enhancement necessitates a difficult balance in the assay's dynamic range-an issue that persisted throughout the various screening efforts.

### 2. Exploration of Modulation Characteristics and Trends Across PRR Agonists

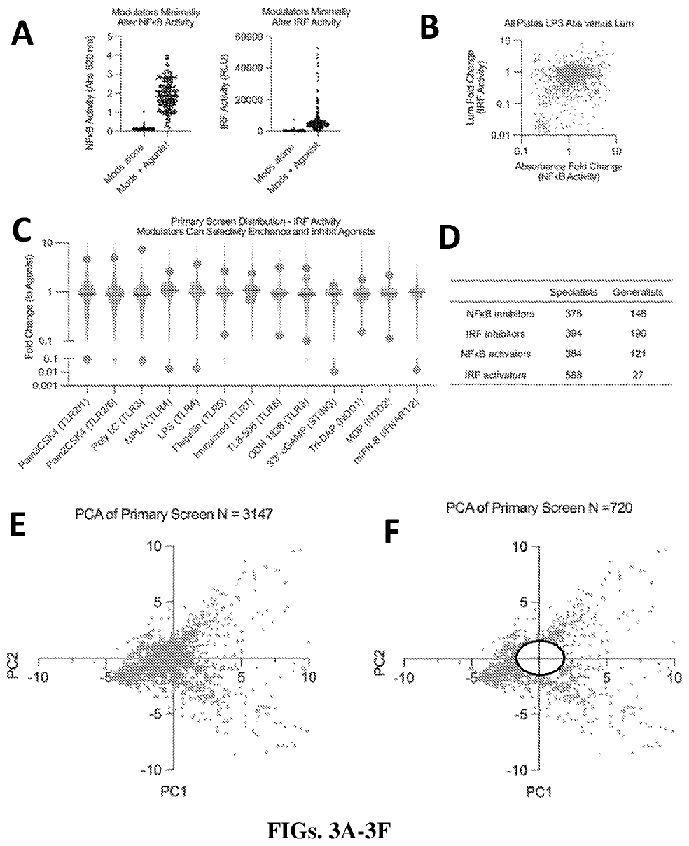

With a large dataset in place, the inventors began to study modulator activity—with a specific interest in both pathway crosstalk and individual receptor trends. First, the inventors ensured that modulators alone do not exhibit inherent stimulation of either NF-κB or IRF, but rather that only modulator and agonist combination leads to variation in transcription factor activity (FIG. 3A). Compounds with significant activation of either pathway were removed from further study, but these were less than 1% of the total library. Upon comparing NF-κB and IRF transcription activity, the inventors observed little correlation between the two, indicating these pathways can be studied independently with the assay (FIG. 3B). Additionally, the inventors observed that modulators acted specifically or generally over multiple PRRs. For example, modulator X may only enhance IRF for TLR4 while other PRRs' activity remain unaffected. Conversely, modulator Y may enhance IRF for all receptors. To identify each type of modulation, the inventors term immunomodulators that are specific to one or two receptors “specialists” and modulators that affect all or almost all receptors “generalists” (FIG. 3C). Further, some modulators can be enhancers of one PRR for a particular pathway and yet inhibitors of another PRR for the same pathway. The inventors see wide distributions across each receptor, with some agonists showing greater statistical significance due to a larger dynamic range. Monitoring distributions across similar PRR targets reveals a correlation in their activity. For instance, MPLA and LPS, both TLR4 agonists, show similar trends across NF-κB and IRF activity. Pam2CSK4 (TLR1/2), Pam3CSK4 (TLR2/6), and other NF-κB dominant agonists also have degrees of correlation (FIG. 8).

### 3. Down Selection Via Removal of Inactive and Undesirable Combinations

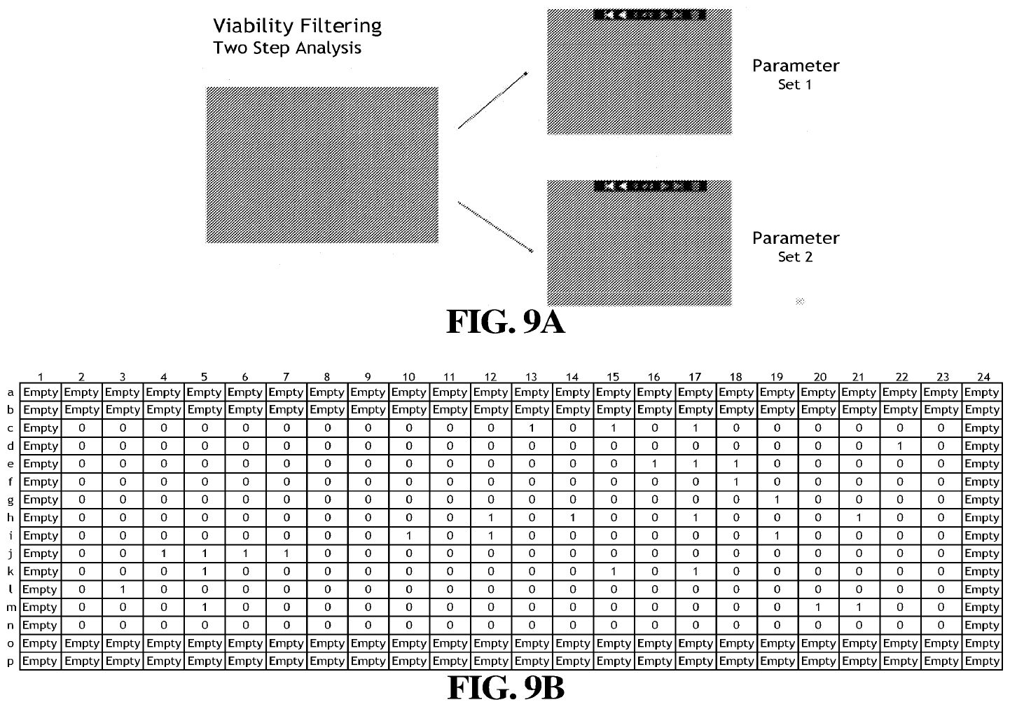

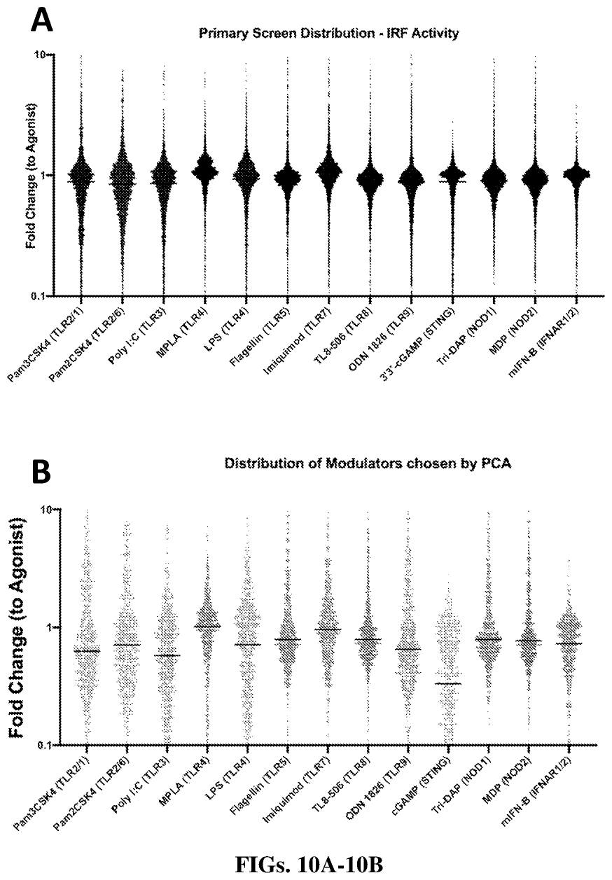

With results from these initial screens, the inventors sought to identify high value compounds and remove inactive and toxic modulator/agonist combinations. The inventors' planned secondary screen implement assays which demand increased time and cost, and thus require a down selection in the number of compounds to be studied. The inventors first designed a high throughput method to filter toxic modulators by measuring viability. As a proxy of viability, the inventors used live cell imaging combined with digital analysis to create confluency masks (FIG. 9) (20). Employing this method allowed the inventors to monitor toxicity on the same experimental data plates the inventors collected IRF and NF-κB data from. The inventors determined toxic compounds as those with <70% the viability of the inventors' negative, unstimulated controls. The inventors confirmed the use of confluency as an indicator of cell health with a secondary dedicated viability assay, CellTiter-Glo (21).

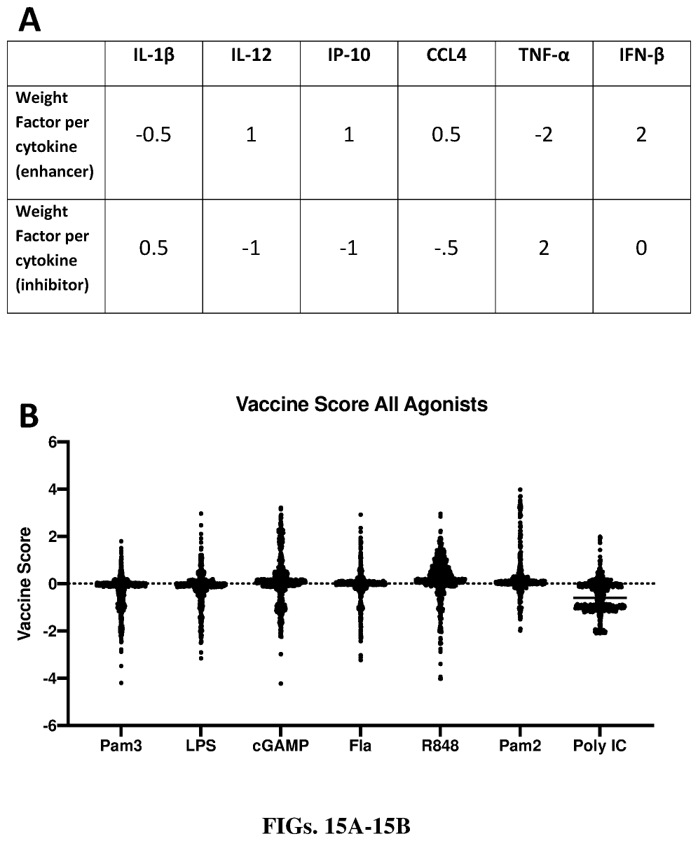

High throughput screening utilizes Z-factors as a proxy for statistical reliability of an assay, due to the dynamic range of the positive controls and the inherent pathways for each agonist, a Z-factor score greater than zero was not obtained for certain agonists/transcription factor combinations. After applying the viability mask, the inventors identified compounds with the highest likelihood of altering IRF and NF-κB responses. The inventors removed specific weaker agonists that only showed basal activity based on a lower Z-factor cutoff score (Table 3). In this down selection step, the inventors did not prioritize enhancement or inhibition of either pathway, but instead sought only to remove compounds that had minimal effects on PRR agonist activity. Thus, the inventors ran principal component analysis on the dataset to quantitatively compare levels of variance between the compounds (22). This dataset included both NF-κB and IRF distributions from eight agonists for a total of 16 variables. PC1 and PC2 accounted for 49% of the variation within the inventors' dataset. By selecting compounds which varied only more than a circle with a radius of ˜1.75 PCA units centered on the origin, the inventors reduced effective immunomodulators from 3,147 compounds to 720 modulators (FIG. 3F). This cutoff was chosen based off the screening limitations for modulators the inventors can explore in the next screen owing to the limited ability to screen for multiplexed cytokines. The inventors created this cutoff with the goal of keeping as many modulating compounds as possible that the inventors can test in a lower throughput secondary screen. The PCA chosen compounds retained the highest and lowest activity of the primary screen distribution while successfully removing modulators that did not alter the response of the cells (FIG. 15).

TABLE 3
Z-factor analysis of Primary Screen agonists

| | | | NF-κB | | IRF |
|---|---|---|---|---|---|
| Agonist | Target | NF-κB | Z-factor | IRF | Z-factor |
| Pam3CSK4 | TLR 2/1 | x | 0.193448 | | <0 |
| Pam2CSK4 | TLR 2/6 | x | 0.509904 | | <0 |
| Poly I:C | TLR3 | | <0 | x | <0 |
| MPLA | TLR4 | x | 0.729615 | x | 0.668819 |
| LPS | TLR4 | x | 0.330641 | x | 0.425865 |
| Flagellin | TLR5 | | <0 | | <0 |
| Imiquimod | TLR7 | x | 0.826412 | | <0 |
| TL8-506 | TLR8 | | <0 | | <0 |
| ODN 1826 | TLR9 | x | 0.785698 | x | <0 |
| 3′3′-cGAMP | STING | | <0 | x | 0.699694 |
| Tri-DAP | NOD1 | | <0 | | <0 |
| MDP | NOD2 | | <0 | | <0 |
| mIFN-β | IFNAR1/2 | | <0 | x | <0 |

4. Secondary Libraries Show Modulation cross a Six-Cytokine Panel

Cytokines and chemokines are a broad category of secreted proteins important for adjuvants in regulating adaptive immunity (23). However, excessive production of cytokines by adjuvants can result in direct tissue damage and are correlated with vaccine tolerability (24, 25). To validate the findings of the primary screening results and to explore the impact on downstream immune outcomes, the inventors measured levels of six cytokines and chemokines involved in inflammation and adaptive responses (IL-12p40, IP-10, IL-1b, CCL4, TNF-α, and IFN-b, Table 4). The inventors measured cytokine expression through AlphaLISA, an assay well-suited to high throughput due to its large dynamic range, in situ measurement, and broad range of cytokines. It has been previously used to investigate small molecule biological inhibitors (26-28).

As the inventors narrowed the compounds, the inventors wanted to ensure compatibility with human immune responses. Conveniently, human AlphaLISAs had many more multiplex options available than murine counterparts which lowered costs. After initial screening of cytokines from stimulated human THP-1 monocytes, the inventors found weak expression/measurements from some identified targets including IL-27 and IL-6. The inventors' final cytokine panel of IL-12p40, IP-10, IL-1b, CCL4, TNF-α, and IFN-b was chosen for their involvement in vaccine responses, excellent dynamic range and assay metrics (Table 4) (29). TNF-α, IL-1b, and IL-6 are endogenous pyrogens as there are multiple reports correlating them with induction of fever in vaccine tolerability (30). TNF-α was chosen as it is a robust correlate for inflammation and is one of the most studied cytokines. IL-1b is produced by activated macrophages and is another mediatory of inflammatory response and can induce expression of other interleukins by γδT cells (31). IL-1b is a measure of inflammation that is partially outside direct NF-κB regulation, unlike TNF-α, which will enable us to decipher by which pathway compounds may bias inflammation. The inventors chose to study IFN-b as it strongly correlates with IRF pathway results from the inventors' primary screen. IFN-b is a well-characterized antiviral type I interferon that induces cells to make IFN-α—amplifying the interferon response (32). IL-12/23p40 activates NK cells and induces production of IFN-g (33). IP-10 (CXCL10) is a chemoattractant for T cells and DC cells and correlated in adjuvants studies with an early signal of induction of strong responses (34). Finally, CCL4 is a chemoattractant for monocytes and NK cells (35). For example, IP-10 and CCL4 were recently shown to correlate with positive response in the BNT162b2 immune response whereas excess TNF-α or IL-6 can correlate with tolerability issues (36).

TABLE 4
Cytokines measured. Functions of cytokines chosen
for secondary screen in AlphaPlex investigation

| Cytokine | Function |
|---|---|
| TNF-α | Promotes inflammation |
| IL-1β | Fever, partially outside NF-κB |
| IFN-β | Antiviral, increased MHCI |
| IL-12/23 (p40) | Activates NK cells, induces IFN-γ, roles in T-cell |
| | polarization |
| IP-10 (CXCL10) | Chemoattractant for T cells, DCs |
| CCL4 | Chemoattractant for monocytes, NK cells, others |

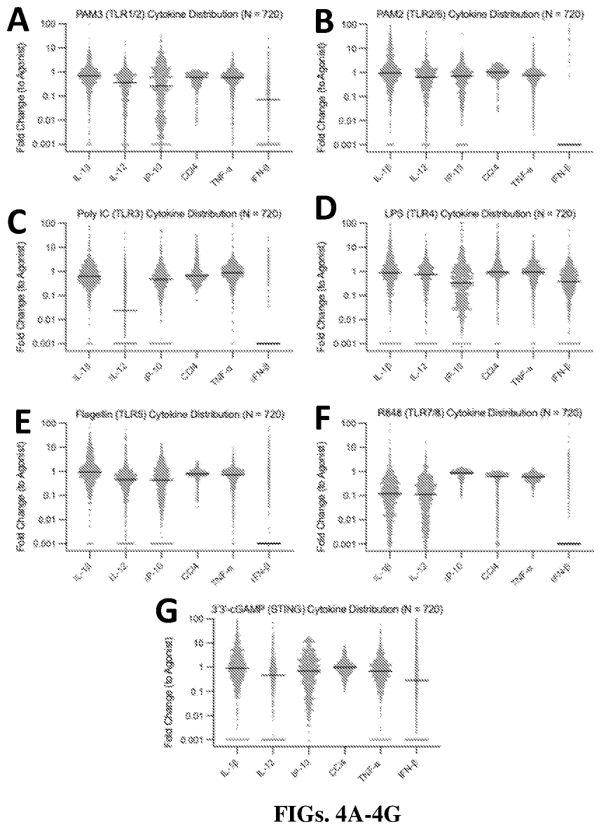

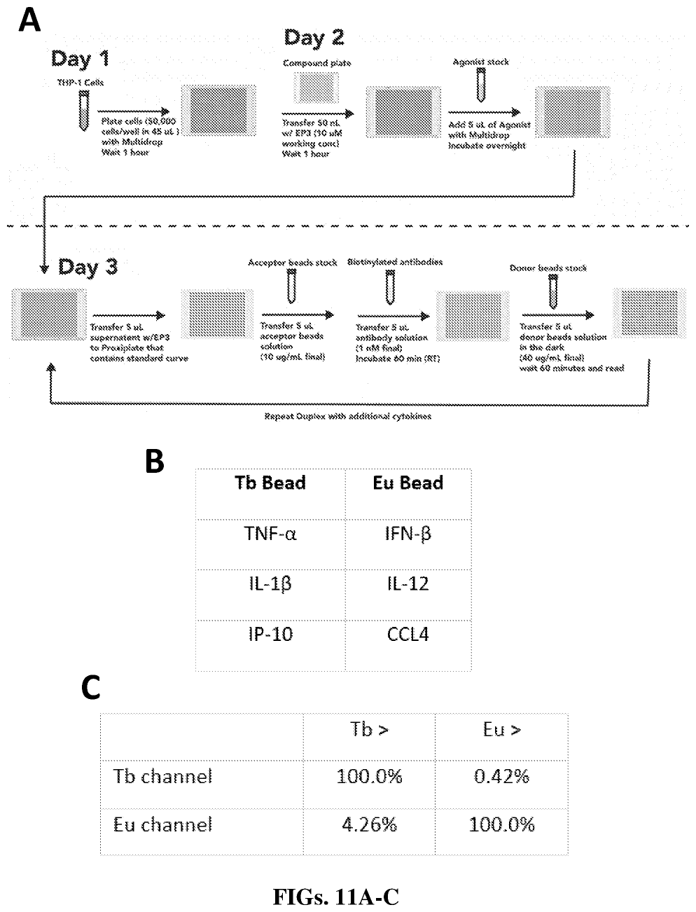

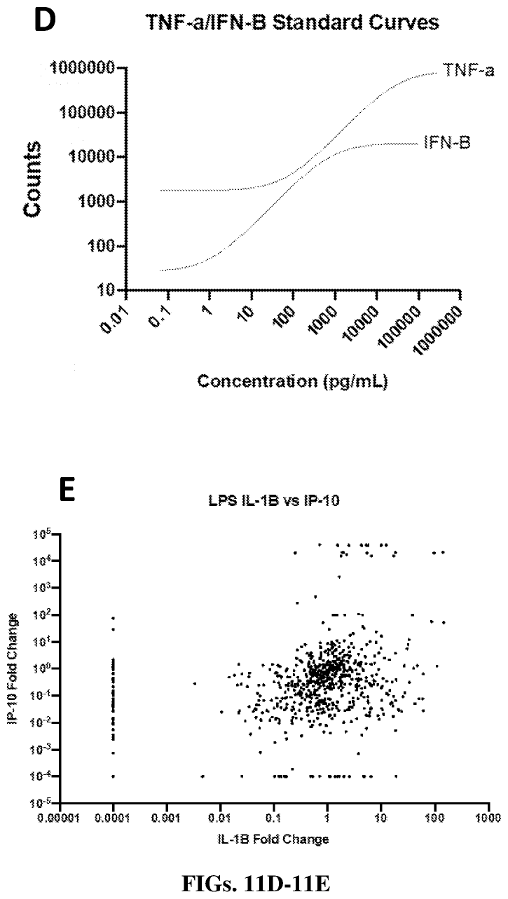

The secondary assay had an identical workflow as the primary screen prior to the cytokine assay. Cell supernatants were collected and cytokines were measured in three duplex measurements (FIG. 11A). Similarly, the inventors optimized standard curve ranges, crosstalk correction factors, incubation times and other factors of the secondary screen (FIGS. 11B-11D). As in the inventors' primary screen, modulators enhance or inhibit cytokine production among all six cytokines and for each agonist independently (FIGS. 4A-4G, FIG. 11E). The distributions within cytokines vary based on the dynamic range and Z-factor obtained for each cytokine and agonist studied (Table 5). High throughput screening utilizes Z-factors as a proxy for statistical reliability of an assay, due to the dynamic range of the positive controls and the inherent cytokines secreted for each agonist, a Z-factor score greater than zero was not obtained for certain agonists/transcription factor combinations. Since each agonist produced differing levels of cytokine that were sometimes at the limits of standard curve, amending this assay to a high throughout nature was challenging. Because the assay beads were multiplexed, dilution of individual wells or selection of other AlphaPlex excitation/emission profiles would increase cost and time significantly. This resulted in some skewing of cytokines with lower or higher response levels. Most notably, IFN-b which had a relatively low signal and concentration, and IP-10/CCL4 which had high signal and concentrations. Since the inventors measured fold-change and reduce dimensionality further in analysis, this approach was adequate to compare compounds within the inventors' dataset for down-selection.

TABLE 5
Z-factor analysis of secondary screen

| | | Conc | Cytokine | | | | | |
|---|---|---|---|---|---|---|---|---|
| Agonist | Target | (μg/mL) | IL-1β | IL-12 | IP-10 | CCL4 | TNF-α | IFN-β |
| Pam3CSK4 | TLR2/1 | 0.1 | .51 | .67 | .23 | .82 | .68 | — |
| Pam2CSK4 | TLR2/6 | 0.1 | −.76 | .69 | .12 | .62 | .66 | — |
| LPS | TLR4 | 0.1 | .69 | .32 | .50 | .67 | .71 | — |
| Flagellin | TLR5 | 1 | .03 | .60 | −.19 | .57 | .71 | — |
| R848 | TLR7 | 10 | .79 | .88 | .87 | .56 | .61 | −.6 |
| 3′3′-cGAMP | STING | 10 | — | — | — | — | −.69 | — |
| Poly IC | TLR3 | 1 | — | — | — | — | — | — |

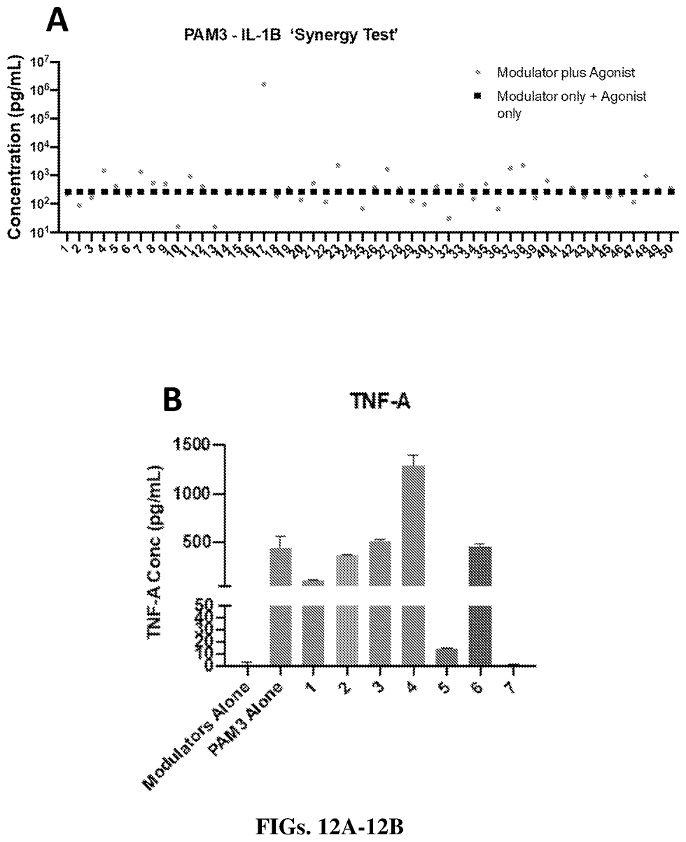

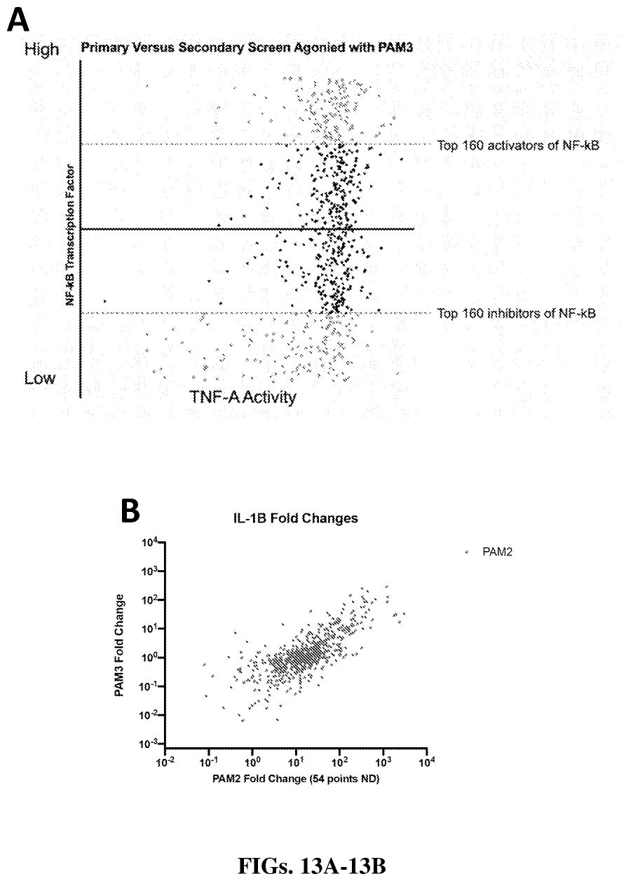

Similar to the initial screen, the inventors observed that modulators alone do not natively affect or induce cytokine release, but rather the combination of agonist and modulator elicits large increases or decreases in cytokine and chemokine production (FIG. 12). While changes in cytokine activity do not always correlate with a corresponding level of change in the transcription factor activity for all modulators, the inventors did observe that, for the most active compounds, transcription factor activity did correlate with an increase of decrease cytokine response. For example, the strongest inhibitors of NF-κB also resulted in the lowest TNF-α levels (FIG. 13A). Also of note, while the modulators can alter responses in unique patterns, they appear to do so with a conservation of pathways to some extent. When comparing agonists with similar profiles such as Pam2CS4K and Pam3CSK4, the inventors observe they share similar trends of enhancement and inhibition for each cytokine (FIG. 13B).

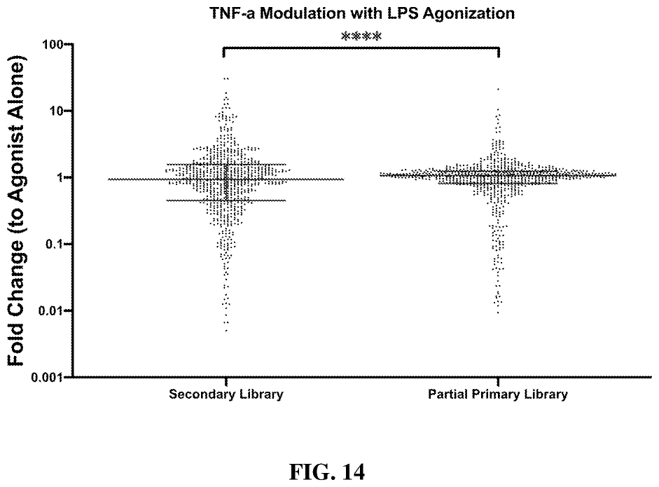

To validate that the down-selection from the primary screen was working, the inventors compared cytokine modulation between the selected compounds and an equivalent, random portion of the original primary screen library. The inventors selected LPS as the agonist owing to wide dynamic range. The secondary, down-selected library contained much more active compounds compared to the random, equal in number primary screen compounds (FIG. 14) supporting that using NF-κB and IRF activity is a valuable down-selection tool.

### 5. Developing a Flexible, Quantitative Scoring System for Top Candidate Selection

With an increasing number of variables to consider when searching for desirable agonist/modulator combinations, the inventors sought to develop a general framework to assist in the final down selection of modulators for testing in various in vivo applications. Since the inventors' previous work focused on improving adjuvants for prophylactic vaccines, the inventors developed the inventors' first scoring system to identify candidates for this use-creating a “vaccine score” as a quantitative metric.

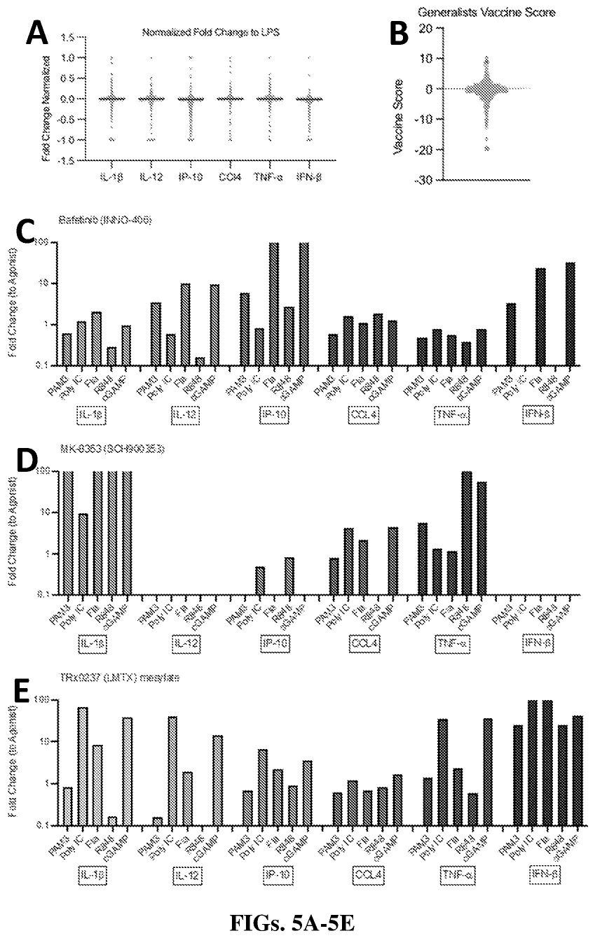

After considering categorical and ranked scoring systems, the inventors decided the best representation of modulator performance would need to preserve cytokine changes but do so by normalizing for each cytokine's and agonist's dynamic range (37). Because of the differences in dynamic range of all the inventors' six cytokines, the inventors sought to normalize the data to ensure no distribution would bias the inventors' results. Thus, the inventors transformed each cytokine's distributions to a range from −1 to 1 (FIG. 5A). Unlike the previous screen, the inventors considered increases and decreases in cytokine responses separately when selecting molecules for vaccination studies. In the vaccine score, a promising candidate would need to produce minimal pro-inflammatory cytokines while increasing IFN-b and chemokine production (24, 25). Additionally, the inventors might prioritize the importance of one cytokine's modulation over another. To account for each of these issues, the inventors assigned weighting variables of varying magnitudes to each cytokine depending on the desired modulatory effect (FIG. 15A) (38). Because modulators acted on individual receptors with distinct responses, the first result from the vaccine scoring system is a “specialist” score for a specific agonist+modulator combination (FIG. 15B). To then identify which modulators have more general trends across multiple PRRs, the individual scores were summed to provide a “generalist” score across all agonists (FIG. 5B).

The result of the vaccine scoring methodology created a spread amongst compounds ranging from 10 at the highest to nearly −20 at the lowest. Within the highest rated compounds for generalist modulators, the scoring system resulted in approximately 20 with scores between 6-10 from the total pool of 720 potential compounds. Amongst these, the inventors included one example compound to demonstrate the patterns observed for the individual modulators—Bafetinib (Inno-416) (FIG. 5C). Bafetinib was one of the highest scoring compounds on the inventors' ranking system. Examining this further by individual cytokines, the inventors saw its remarkable enhancement of IP-10 across almost all agonists and enhancement of IFN-b and IL-12 for several agonists were factors that increased its scores. Similarly, Bafetinib decreased TNF-α expression across all agonists with notably high suppression for Pam3 and resiquimod (R848). Perhaps most critical, however, is that in selecting for a “generalist”, the inventors note that it is not equally effective with every agonist. Comparing its enhancement of IP-10 and IFN-b for Pam3, Pam2 vs cGAMP, there are nearly 2 orders of magnitude in difference between its enhancement ability for these agonists and pathways. This suggests that depending on the application, a generalist might still have limitations vs specialist modulators for enhancing specific pathways. However, for suppression of inflammatory signals and thereby tolerability, this example and many of the modulators the inventors studied did appear to be broadly general. Suppressing inflammatory signals, even partially, across many categories of receptors suggest that generalists might be better applied toward improved tolerability and broad use in improved vaccination.

The inventors can contrast Bafetinib's results with MK-8353 (FIG. 5D), which is a compound in the dataset with a strong negative vaccine score. This compound ablates IL-12, IFN-b, and IP-10 across nearly all receptors studied while simultaneously enhancing IL-1b and TNF-α to up to 100 fold. MK-8353 will not be useful as a vaccine adjuvant, but it's ability to radically alter secreted cytokines highlights the wide-ranging potential of the inventors' modulators. This score is tailored to prophylactic vaccine adjuvants, but compounds within this dataset may be applicable for exploration in additional applications. For instance, TRx0237(LMTX) mesylate (FIG. 5E), may warrant additional study in a cancer immunotherapy as it upregulates beneficial antitumor cytokines and chemokines (39, 40). This compound can enhance TNF-α up to 36-fold using cGAMP and is shown to enhance IFN-b secretion across all agonists studied.

The inventors used the general vaccine score to identify lead modulators of interest that the inventors wanted to carry forward for preliminary in vivo studies. The foundation and simple mathematical nature of the inventors' scoring system allows one to apply this methodology to different areas of interest, albeit limited by the cytokine panel studied. Additionally, further screening efforts such as cell surface marker expression could be incorporated into this scoring system in the future. While the inventors created a vaccine scoring system, a similar methodology could be tailored toward other applications, whether that is for inflammation therapeutics or cancer immunotherapy, control of immune pathways has potential in many immune-therapeutic spaces.

### 6. Identified Candidates Show Promising Results in Murine Vaccination Studies

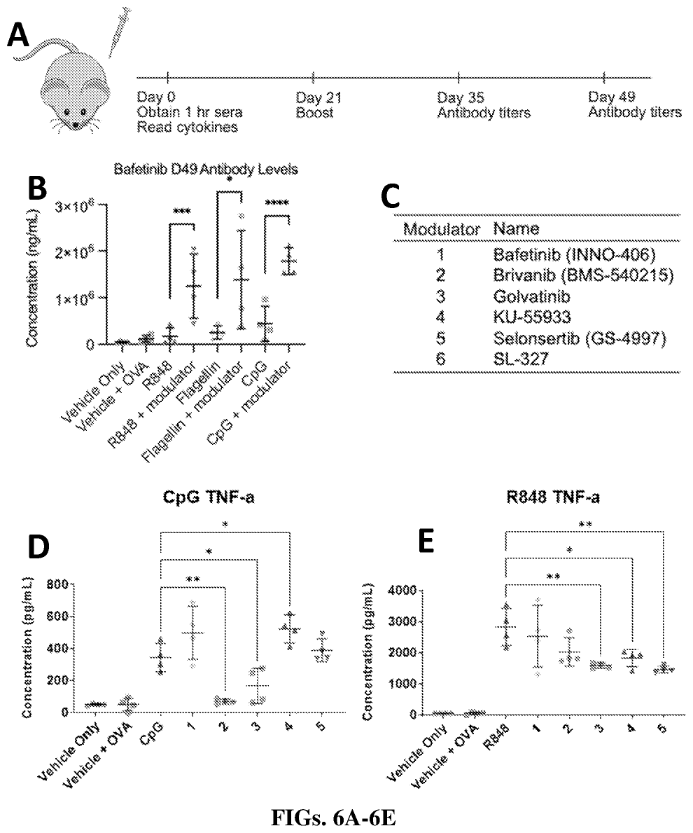

The inventors used the vaccine score to select modulators to test in a murine in vivo model. The inventors repeated the traditional prime-boost vaccination schedule as in the inventors' previous preliminary studies using ovalbumin as a model antigen (9, 10). To test the generalist nature of the modulators, these subunit vaccinations were adjuvanted with a subset of the PRR agonists from the inventors' preliminary screen: R848 (TLR7/8), flagellin (TLR5), and CpG 1826 (TLR9). This subset was selected both for the previous use in vaccines and for a broader cross section of potential use in both subunit (R848, CpG) and as an approximation of whole bacterial (flagellin, CpG) vaccine products (41-43). The inventors chose a modulator dosage of 1.5 μmol, guided by the inventors' previous experience with small molecules and compounds solubility limitations (10). To improve formulation of these hydrophobic compounds, the inventors used a 1:1 mixture of DMSO: Adda Vax as a vehicle. The inventors monitored inflammatory cytokine levels one hour following initial injections and monitored antigen specific antibody levels at the indicated timepoints post-boost (FIG. 6A). While AddaVax has inherent adjuvanting properties, the inventors determined its use appropriate because (a) the experiment was testing the modulation of pre-existing adjuvants which would be need to be formulated in their administration, and (b) the inventors could control for inherent alterations to their response via separate adjuvant only (no modulator) controls.

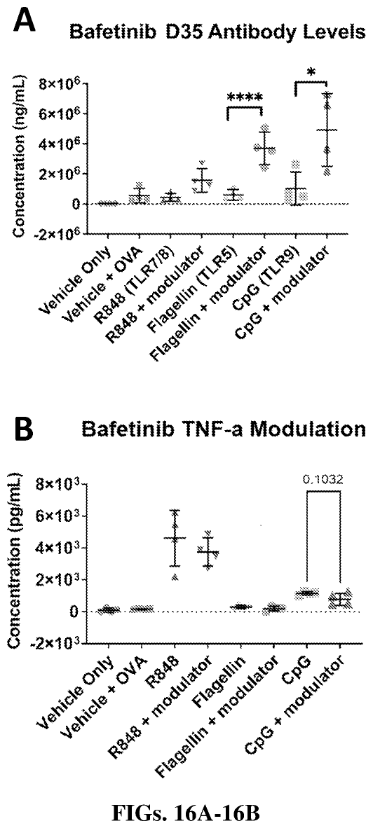

The goal, as previously, was to find compounds that would (a) increase tolerability of the vaccine formulation which the inventors approximate using a simple metric of systemic cytokines 1 hr after injection and (b) improve antibody responses to the vaccination. The inventors began by experimenting with Bafetinib, the highest performing compound from the generalist scoring system. The addition of this modulator resulted in an increased the humoral response for all three agonists tested (FIG. 6B). Addition of Bafetinib improved the antibody response for these adjuvants ranging from 2 to 6 fold over their agonist/antigen controls. This increase was remarkably consistent across all the agonists and persisted across both time points. (FIG. 16A). The inventors did not, however, observe significant cytokine modulation with this modulator (FIG. 16B).

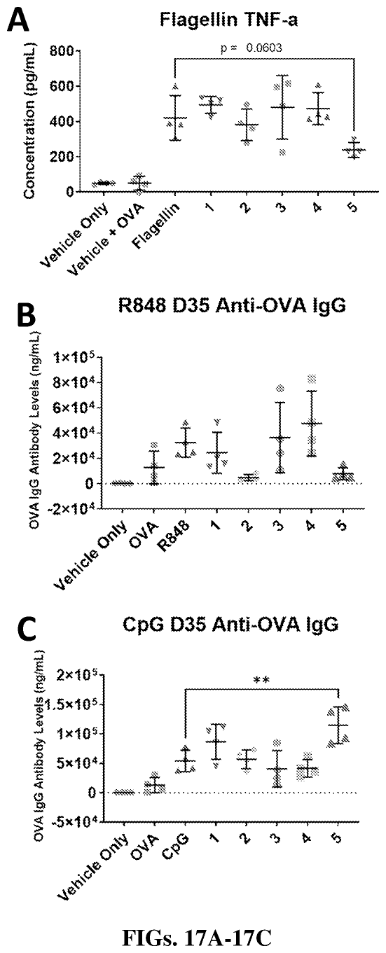

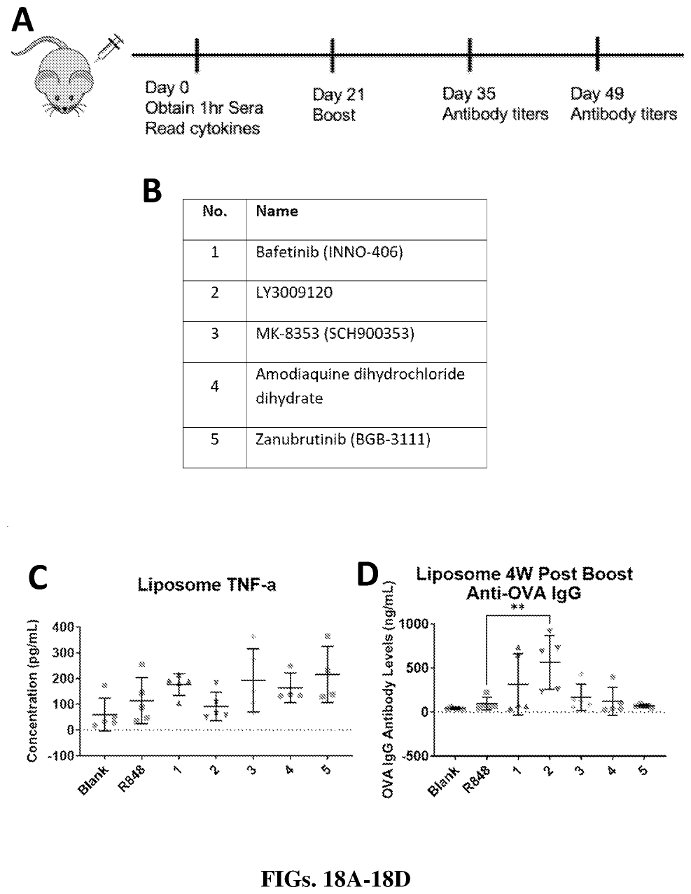

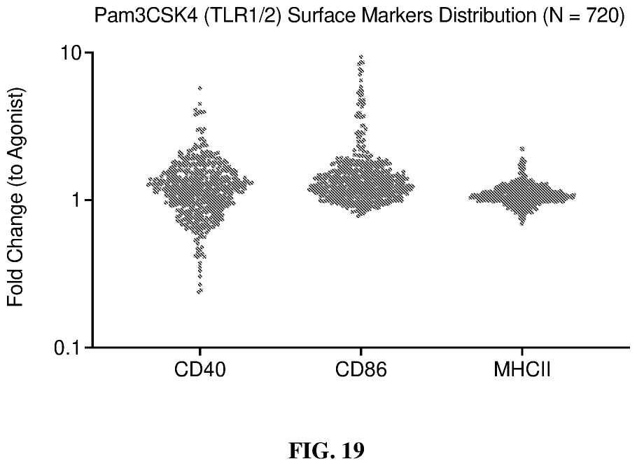

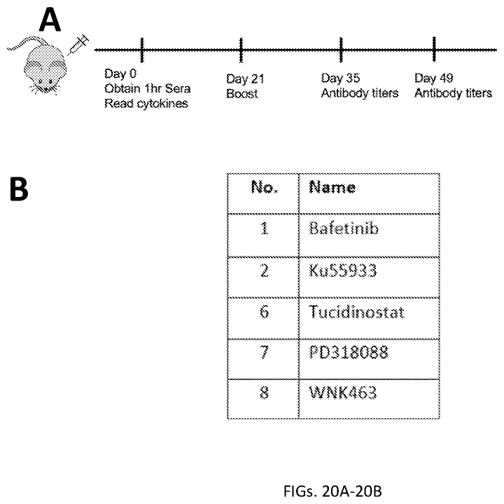

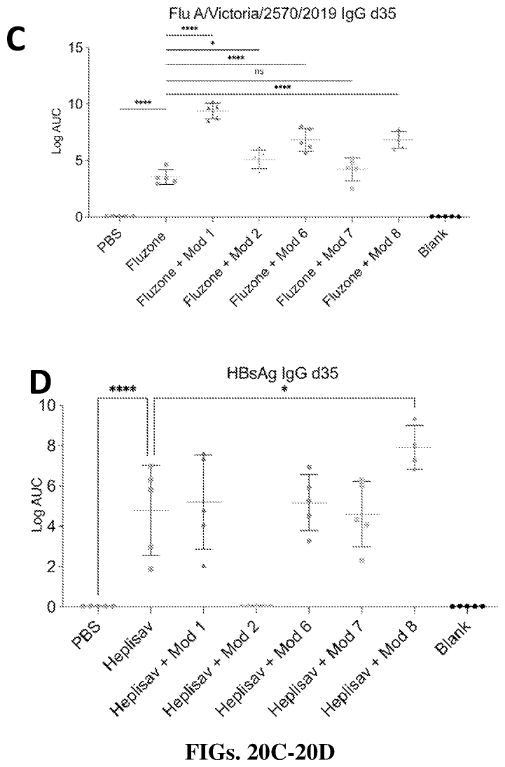

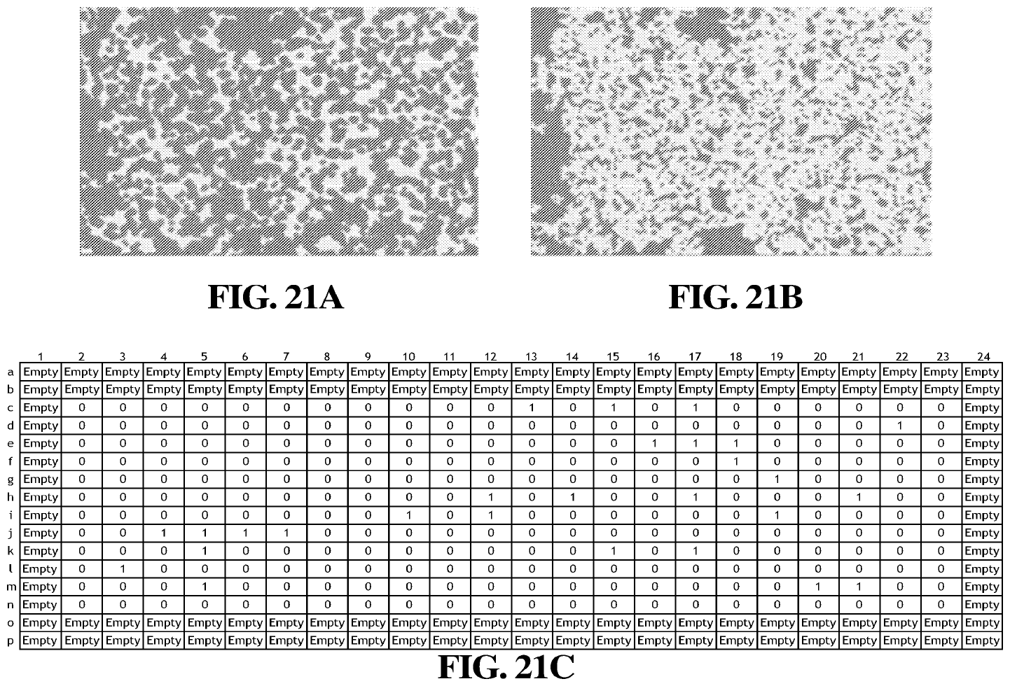

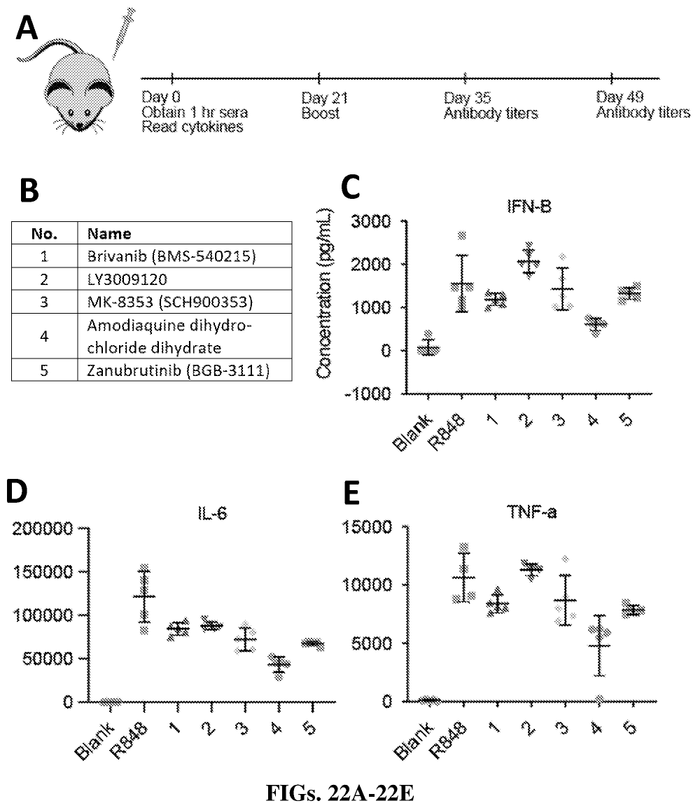

Emboldened by these positive results, the inventors expanded the search to 5 more high scoring generalists following the same vaccination schedule as before (FIG. 6C). The inventors observed that for the 5 modulators, compounds 2 & 3 strongly decreased TNF-α for CpG to near 0 at 1 hr (FIG. 6D). This level of decrease is strongly correlated with improvement in clinical scoring, temperature drop, and weight-loss providing strong indications that these compounds could be used to improve the tolerability of CpG in further applications. Interestingly, there were similarities between which compounds reduced inflammatory cytokines for CpG and for R848. However, as with the inventors' previous experiments, modulators were unable to completely remove the inflammatory nature of R848 owing to is more rapid diffusion (9). Yet compound 5 still reduced the inflammation to half the original formulation. No compounds showed significant reduction in cytokines for flagellin, though compound 5 showed a large decrease (FIG. 17A).

The reduction in TNF-α, however, largely did not correlate to a change in antibody levels (FIG. 17B).

To the inventors' surprise and in contrast to the inventors' previous work, the two arms of the inventors' work—systemic cytokine modulation and antibody responses—did not seem to be inherently coupled. Bafetinib, the modulator that enhanced antigen-specific IgG levels the most only, partially downregulated inflammatory cytokines depending on the adjuvant. Conversely, modulators that did alter cytokine levels were minimally impactful on the humoral response. However, the inventors identified both modulators that strongly decreased the systemic cytokines of current adjuvants and modulators which enhanced antibody responses. After multiple rounds of investigation, the inventors have identified two classes of lead modulators: inflammatory cytokine reducing modulators as well as IgG antibody enhancing modulators.

7. Development of Novel mRNA Vaccination System; Immunomodulators Suppress In Vitro and In Vivo Inflammatory Responses by mRNA Vaccine

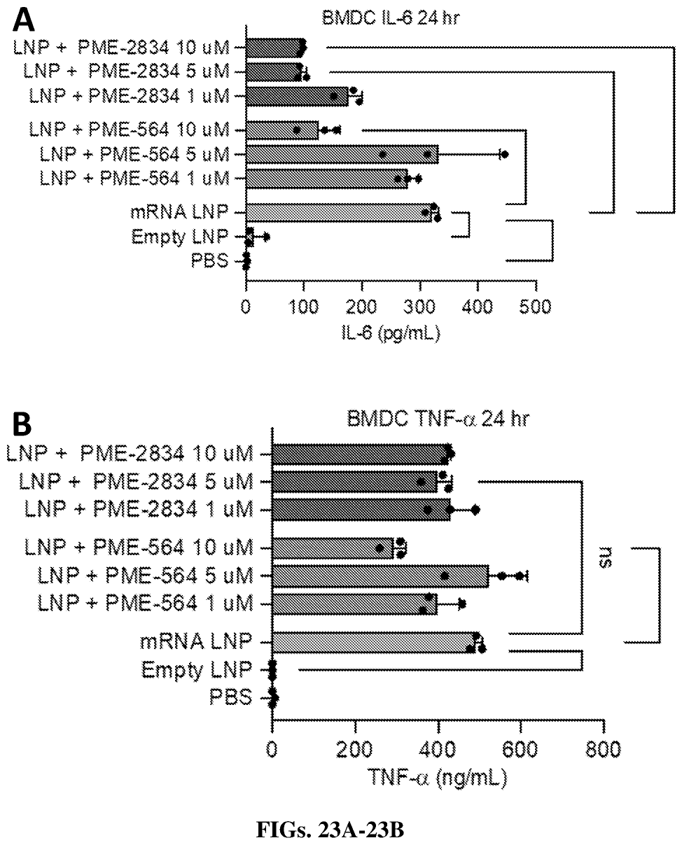

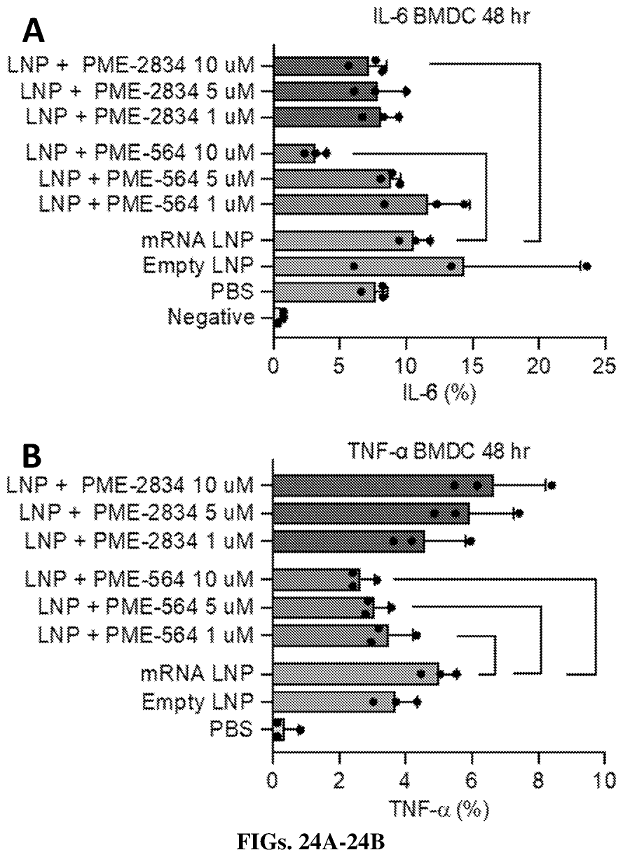

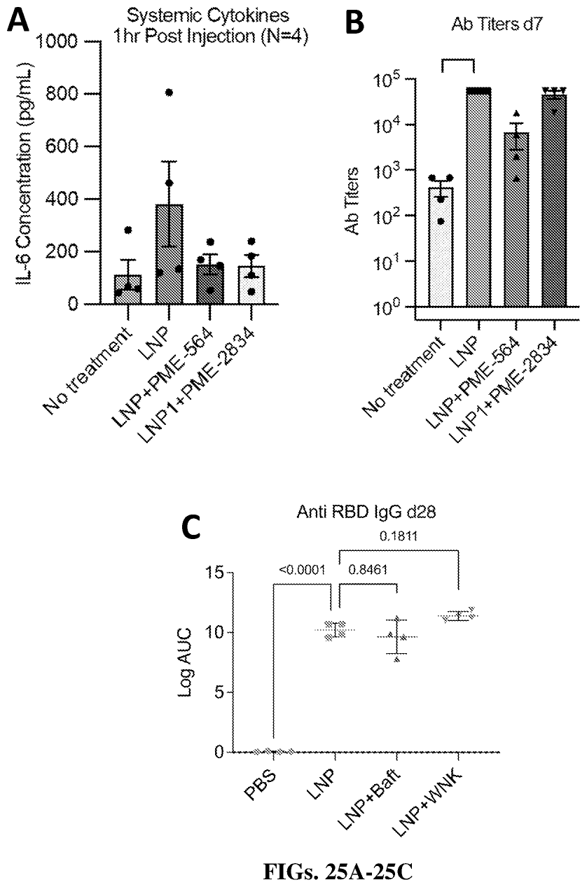

Experiments were conducted to determine whether immunomodulators suppress inflammatory response of mRNA vaccine in vitro and in vivo. For in vitro experiments, immunomodulators at various concentrations were added to BMDCs one hour before adding mRNA vaccine. Supernatant systemic cytokines were measured at 24 hours (FIGS. 23A-23B) and intracellular cytokine staining was performed at 48 hours (FIGS. 24A-24B). PME-564 and PME-2834 significantly reduced TNF-α and IL-6 production at both timepoints. For in vivo experiments, C57BL/6J (n=4) were immunized intramuscularly with home-made SARS-COV-2 mRNA vaccine (WA-1/Omicron, 5 μg/dose) and immunomodulators (1.5 μmol). Immunomodulators also reduced the production of IL-6 cytokine (despite being statistically insignificant) (FIG. 25A). Vaccination was unable to statistically decrease production of systemic cytokines (FIG. 25A). LNPs reduce their stability upon addition of DMSO, and thus far PME-564 is most soluble in DMSO and other organic solvents. Additional synthetic modifications to PME-564 to allow encapsulation into the LNPs will be explored in future. Simultaneously, antibody titers against SARS-COV-2 spike were measured post day 7 post injection. High antibodies were found in all vaccinated groups. Particularly, there were no difference between ‘mRNA vaccine alone’ and either modulator group (FIG. 25B). Overall, these in vitro and in vivo studies suggest that immunomodulators can potentially reduce excessive inflammatory responses by the mRNA vaccine without decreasing antibody titers.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and those cited elsewhere herein, to the extent that they provide exemplary procedural or other details supplementary to those set forth herein, are specifically
1. Y. Perrie, A. R. Mohammed, D. J. Kirby, S. E. McNeil, V. W. Bramwell, Vaccine adjuvant systems: enhancing the efficacy of sub-unit protein antigens. Int J Pharm 364, 272-280 (2008).
2. D. T. O'Hagan, E. De Gregorio, The path to a successful vaccine adjuvant—‘the long and winding road’. Drug Discov Today 14, 541-551 (2009).
3. A. M. Harandi, G. Davies, O. F. Olesen, Vaccine adjuvants: scientific challenges and strategic initiatives. Expert Review of Vaccines 8, 293-298 (2009).
4. D. H. Munn, V. Bronte, Immune suppressive mechanisms in the tumor microenvironment. Curr Opin Immunol 39, 1-6 (2016).
5. T. Liu, L. Zhang, D. Joo, S.-C. Sun, NF-κB signaling in inflammation. Sig Transduct Target Ther 2, 17023 (2017).
6. E. Platanitis, T. Decker, Regulatory Networks Involving STATs, IRFs, and NFκB in Inflammation. Frontiers in Immunology 9 (2018).
7. C. Olive, Pattern recognition receptors: sentinels in innate immunity and targets of new vaccine adjuvants. Expert Review of Vaccines 11, 237-256 (2012).
8. T. Kawai, S. Akira, The role of pattern-recognition receptors in innate immunity: update on Toll-like receptors. Nat Immunol 11, 373-384 (2010).
9. B. A. Moser, et al., Increased vaccine tolerability and protection via NF-κB modulation. Sci. Adv. 6, eaaz8700 (2020).
10. B. A. Moser, et al., Small Molecule NF-κB Inhibitors as Immune Potentiators for Enhancement of Vaccine Adjuvants. Front Immunol 11, 511513 (2020).
11. Y. Tanaka, Z. J. Chen, STING specifies IRF3 phosphorylation by TBK1 in the cytosolic DNA signaling pathway. Sci Signal 5, ra20 (2012).
12. N. S. Ahmed, et al., BRD9 regulates interferon-stimulated genes during macrophage activation via cooperation with BET protein BRD4. Proc Natl Acad Sci USA 119, e2110812119 (2022).
13. T. Kawasaki, T. Kawai, Toll-Like Receptor Signaling Pathways. Frontiers in Immunology 5 (2014).
14. C. B. Maddox, L. Rasmussen, E. L. White, Adapting Cell-Based Assays to the High Throughput Screening Platform: Problems Encountered and Lessons Learned. JALA Charlottesv Va 13, 168-173 (2008).
15. W. F. An, N. Tolliday, Cell-Based Assays for High-Throughput Screening. Mol Biotechnol 45, 180-186 (2010).
16. A. C. D. Salyer, et al., Identification of Adjuvantic Activity of Amphotericin B in a Novel, Multiplexed, Poly-TLR/NLR High-Throughput Screen. PLOS ONE 11, e0149848 (2016).
17. Y. Guan, K. Omueti-Ayoade, S. K. Mutha, P. J. Hergenrother, R. I. Tapping, Identification of Novel Synthetic Toll-like Receptor 2 Agonists by High Throughput Screening *. Journal of Biological Chemistry 285, 23755-23762 (2010).
18. L. Martínez-Gil, et al., Identification of Small Molecules with Type I Interferon Inducing Properties by High-Throughput Screening. PLOS ONE 7, e49049 (2012).
19. D. L. Burdette, et al., STING is a direct innate immune sensor of cyclic di-GMP. Nature 478, 515-518 (2011).
20. F. Haenel, N. Garbow, Cell Counting and Confluency Analysis as Quality Controls in Cell-Based Assays. Perkin Elmer, 5 (2014).
21. A. Single, H. Beetham, B. J. Telford, P. Guilford, A. Chen, A Comparison of Real-Time and Endpoint Cell Viability Assays for Improved Synthetic Lethal Drug Validation. J Biomol Screen 20, 1286-1293 (2015).
22. J. Lever, M. Krzywinski, N. Altman, Principal component analysis. Nature Methods 14, 641-642 (2017).
23. G. Ramesh, A. G. MacLean, M. T. Philipp, Cytokines and chemokines at the crossroads of neuroinflammation, neurodegeneration, and neuropathic pain. Mediators Inflamm 2013, 480739 (2013).
24. L. M. Christian, K. Porter, E. Karlsson, S. Schultz-Cherry, Proinflammatory cytokine responses correspond with subjective side effects after influenza virus vaccination. Vaccine 33, 3360-3366 (2015).
25. W. L. Simon, H. M. Salk, I. G. Ovsyannikova, R. B. Kennedy, G. A. Poland, Cytokine production associated with smallpox vaccine responses. Immunotherapy 6, 1097-1112 (2014).
26. M. Bielefeld-Sevigny, AlphaLISA Immunoassay Platform-The “No-Wash” High-Throughput Alternative to ELISA. ASSAY and Drug Development Technologies 7, 90-92 (2009).
27. X. Tang, et al., A high-throughput screening method for small-molecule inhibitors of the aberrant mutant SOD1 and dynein complex interaction. J Biomol Screen 17, 314-326 (2012).
28. L. Prabhu, et al., Development of an AlphaLISA high throughput technique to screen for small molecule inhibitors targeting protein arginine methyltransferases. Mol Biosyst 13, 2509-2520 (2017).
29. W. Chanput, J. J. Mes, H. J. Wichers, THP-1 cell line: An in vitro cell model for immune modulation approach. International Immunopharmacology 23, 37-45 (2014).
30. C. A. Dinarello, Cytokines as Endogenous Pyrogens. The Journal of Infectious Diseases 179, S294-S304 (1999).
31. K. Ren, R. Torres, Role of interleukin-1β during pain and inflammation. Brain Res Rev 60, 57-64 (2009).
32. T. Fujita, et al., Induction of the transcription factor IRF-1 and interferon-beta mRNAs by cytokines and activators of second-messenger pathways. Proc Natl Acad Sci USA 86, 9936-9940 (1989).
33. The interleukin 12 p40 gene promoter is primed by interferon gamma in monocytic cells. J Exp Med 183, 147-157 (1996).
34. Recombinant human interferon-inducible protein 10 is a chemoattractant for human monocytes and T lymphocytes and promotes T cell adhesion to endothelial cells. J Exp Med 177, 1809-1814 (1993).
35. S. Sindhu, et al., The Cooperative Induction of CCL4 in Human Monocytic Cells by TNF-α and Palmitate Requires MyD88 and Involves MAPK/NF-κB Signaling Pathways. Int J Mol Sci 20, 4658 (2019).
36. C. Bergamaschi, et al., Systemic IL-15, IFN-γ, and IP-10/CXCL10 signature associated with effective immune response to SARS-COV-2 in BNT162b2 mRNA vaccine recipients. Cell Rep 36, 109504 (2021).
37. N. Malo, J. A. Hanley, S. Cerquozzi, J. Pelletier, R. Nadon, Statistical practice in high-throughput screening data analysis. Nat Biotechnol 24, 167-175 (2006).
38. C. Eurtivong, J. Reynisson, The Development of a Weighted Index to Optimise Compound Libraries for High Throughput Screening. Molecular Informatics 38, 1800068 (2019).
39. S. F. Josephs, et al., Unleashing endogenous TNF-αlpha as a cancer immunotherapeutic. Journal of Translational Medicine 16, 242 (2018).
40. T. Mohan, W. Zhu, Y. Wang, B.-Z. Wang, Applications of chemokines as adjuvants for vaccine immunotherapy. Immunobiology 223, 477-485 (2018).
41. D. J. Dowling, Recent Advances in the Discovery and Delivery of TLR7/8 Agonists as Vaccine Adjuvants. IH 2, 185-197 (2018).
42. B. Cui, et al., Flagellin as a vaccine adjuvant. Expert Rev Vaccines 17, 335-349 (2018).
43. C. Bode, G. Zhao, F. Steinhagen, T. Kinjo, D. M. Klinman, CpG DNA as a vaccine adjuvant. Expert Rev Vaccines 10, 499-511 (2011).

1. A method for modulating an immune response comprising administering to a subject an effective amount of:
(a) an adjuvant; and
(b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate, or a combination thereof.

2. A method of immune activation comprising administering to a population of immune cells an effective amount of:
(a) an adjuvant; and
(b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate, or a combination thereof.

3. A method for vaccinating a subject comprising administering to the subject an effective amount of:
(a) an adjuvant;
(b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate, or a combination thereof; and
(c) an antigen.

4. A method for preventing or treating cancer, a bacterial infection, or a viral infection, the method comprising administering to the subject an effective amount of
(a) an adjuvant; and
(b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate.

5. The method of claim 4, wherein the method further comprises administering an antigen.

6. The method of claim 5, wherein the antigen is associated with the cancer, bacteria, or virus.

7. The method of any one of claims 3-6, wherein the antigen is a bacterial antigen, a viral antigen, or a tumor antigen.

8. The method of any one of claims 1-7, wherein (b) comprises or consists of bafetinib (INNO-406).

9. The method of any one of claims 1-8, wherein (b) comprises or consists of LY3009120.

10. The method of any one of claims 1-9, wherein (b) comprises or consists of MK-8353 (SCH900353).

11. The method of any one of claims 1-10, wherein (b) comprises or consists of amodiaquine.

12. The method of any one of claims 1-11, wherein (b) comprises or consists of zanubrutinib (BGB-3111).

13. The method of any one of claims 1-12, wherein (b) comprises or consists of Ku55933.

14. The method of any one of claims 1-13, wherein (b) comprises or consists of Tucidinostat.

15. The method of any one of claims 1-14, wherein (b) comprises or consists of PD318088.

16. The method of any one of claims 1-15, wherein (b) comprises or consists of WNK463.

17. The method of any one of claims 1-16, wherein (b) comprises or consists of TRx0237 (LMTX) mesylate.

18. The method of any one of claims 1-17, wherein the adjuvant comprises 3′3′-cGAMP, Tri-DAP, MDP, and/or mIFN-B.

19. The method of any of claims 1-18, wherein the adjuvant comprises a PRR agonist.

20. The method of claim 19, wherein the PRR agonist is a TLR agonist.

21. The method of claim 20, wherein the TLR agonist is an agonist to TLR1, TLR2/1, TLR2, TLR2/6, TLR3, TLR4, TLR5 TLR7, TLR8, TLR7/8, TLR9, and/or TLR11.

22. The method of claim 20 or 21, wherein the TLR agonist comprises or consists of one or more of peptidoglycan, triacyl lipoproteins, lipoteichoic acid, peptidoglycan from Bacillus subtilis, peptidoglycan from E. coli 0111: B4, peptidoglycan from Escherichia coli K12, peptidoglycan from Staphylococcus aureus, atypical lipopolysaccharide (LPS), Leptospirosis LPS, Porphyromonas gingivalis LPS, a synthetic diacylated lipoprotein, FSL-1, Pam2CSK4, lipoarabinomannan from M. smegmatis, lipomannan from M. smegmatis; triacylated lipoproteins, Pam3CSK4, MALP-2 from mycoplasma, MALP-404 from mycoplasma, Borrelia burgdorferi OspA, Porin from Neisseria meningitidis, Porin from Haemophilus influenza, Propionibacterium acnes antigen mixtures, Yersinia LcrV, lipomannan from Mycobacterium, lipomannan from Mycobacterium tuberculosis, Trypanosoma cruzi GPI anchor, Schistosoma mansoni lysophosphatidylserine, Leishmania major lipophosphoglycan (LPG), Plasmodium falciparum glycophosphatidylinositol (GPI), zymosan, antigen mixtures from Aspergillus fumigatus, antigen mixtures from Candida albicans, antigen mixtures from measles hemagglutinin, double-stranded RNA, polyadenylic-polyuridylic acid (Poly(A:U)), polyinosine-polycytidylic acid (Poly(I:C)), polyinosine-polycytidylic acid high molecular weight (Poly(I:C) HMW), polyinosine-polycytidylic acid low molecular weight (Poly(I:C) LMW)), LPS from Escherichia coli, LPS from Salmonella species, monophosphoryl lipid A, flagellin, flagellin from B. subtilis, flagellin from P. aeruginosa, flagellin from S. typhimurium, single stranded RNAs with 6UUAU repeats, single stranded RNA homopolymer (ssPolyU naked), HIV-1 LTR-derived ssRNA (ssRNA40), ssRNA with 2 GUCCUUCAA repeats (ssRNA-DR)), imidazoquinoline compounds, imiquimod, Imiquimod VacciGrade™, Gardiquimod VacciGrade™, Gardiquimod™, adenine analog CL264, base analog CL307, guanosine analog loxoribine, TL8-506, thiazoquinoline compound CL075, imidazoquinoline compound CL097, 2Bxy, R848, R848 VacciGrade™, CpG ODN, and Toxoplasma gondii Profilin.

23. The method of any one of claims 1-21, wherein the TLR agonist is a TLR7/8 agonist.

24. The method of claim 23, wherein the TLR7/8 agonist is R848.

25. The method of any one of claims 1-24, wherein the TLR agonist is a TLR5 agonist.

26. The method of claim 25, wherein the TLR5 agonist is flagellin.

27. The method of any one of claims 1-26, wherein the TLR agonist is a TLR9 agonist.

28. The method of claim 27, wherein the TLR9 agonist is a CpG oligonucleotide.

29. The method of claim 28, wherein the CpG oligonucleotide comprises CpG 1826.

30. The method of any of claims 1-29, further comprising administering to the subject an additional adjuvant.

31. The method of any one of claims 1-30, wherein the subject is a human subject.

32. The method of any one of claims 1-31, wherein (a), (b), and/or the adjuvant are administered by intramucosal, intramuscular, parenteral, or subcutaneous administration.

33. The method of any one of claims 1-32, wherein the method further comprises administration of a vaccine composition.

34. The method of claim 33, wherein the vaccine composition comprises a flu vaccine, Hepatitis B vaccine, or COVID vaccine.

35. The method of claim 34, wherein the vaccine composition comprises Fluzone or Heplisav.

36. A pharmaceutical composition comprising:
(a) an adjuvant; and
(b) one or more of bafetinib (INNO-406), LY3009120, MK-8353 (SCH900353), amodiaquine, zanubrutinib (BGB-3111), Ku55933, Tucidinostat, PD318088, WNK463, and TRx0237 (LMTX) mesylate, or a combination thereof.

37. The pharmaceutical composition of claim 36, wherein the pharmaceutical composition further comprises an antigen.

38. The pharmaceutical composition of claim 37, wherein the antigen comprises or consists of a bacterial antigen, a viral antigen, or a tumor antigen.

39. The pharmaceutical composition of any one of claims 36-38, wherein (b) comprises or consists of bafetinib (INNO-406).

40. The pharmaceutical composition of any one of claims 36-39, wherein (b) comprises or consists of LY3009120.

41. The pharmaceutical composition of any one of claims 36-40, wherein (b) comprises or consists of MK-8353 (SCH900353).

42. The pharmaceutical composition of any one of claims 36-41, wherein (b) comprises or consists of amodiaquine.

43. The pharmaceutical composition of any one of claims 36-42, wherein (b) comprises or consists of zanubrutinib (BGB-3111).

44. The pharmaceutical composition of any one of claims 36-43, wherein (b) comprises or consists of Ku55933.

45. The pharmaceutical composition of any one of claims 36-44, wherein (b) comprises or consists of Tucidinostat.

46. The pharmaceutical composition of any one of claims 36-45, wherein (b) comprises or consists of PD318088.

47. The pharmaceutical composition of any one of claims 36-46, wherein (b) comprises or consists of WNK463.

48. The pharmaceutical composition of any one of claims 36-47, wherein (b) comprises or consists of TRx0237 (LMTX) mesylate.

49. The pharmaceutical composition of any one of claims 36-48, wherein the adjuvant comprises 3′3′-cGAMP, Tri-DAP, MDP, and/or mIFN-B.

50. The pharmaceutical composition of any of claims 36-49, wherein the adjuvant comprises a PRR agonist.

51. The pharmaceutical composition of claim 50, wherein the PRR agonist is a TLR agonist.

52. The pharmaceutical composition of claim 51, wherein the TLR agonist is an agonist to TLR1, TLR2/1, TLR2, TLR2/6, TLR3, TLR4, TLR5 TLR7, TLR8, TLR7/8, TLR9, and/or TLR11.

53. The pharmaceutical composition of claim 51 or 52, wherein the TLR agonist comprises or consists of one or more of peptidoglycan, triacyl lipoproteins, lipoteichoic acid, peptidoglycan from Bacillus subtilis, peptidoglycan from E. coli 0111: B4, peptidoglycan from Escherichia coli K12, peptidoglycan from Staphylococcus aureus, atypical lipopolysaccharide (LPS), Leptospirosis LPS, Porphyromonas gingivalis LPS, a synthetic diacylated lipoprotein, FSL-1, Pam2CSK4, lipoarabinomannan from M. smegmatis, lipomannan from M. smegmatis; triacylated lipoproteins, Pam3CSK4, MALP-2 from mycoplasma, MALP-404 from mycoplasma, Borrelia burgdorferi OspA, Porin from Neisseria meningitidis, Porin from Haemophilus influenza, Propionibacterium acnes antigen mixtures, Yersinia LcrV, lipomannan from Mycobacterium, lipomannan from Mycobacterium tuberculosis, Trypanosoma cruzi GPI anchor, Schistosoma mansoni lysophosphatidylserine, Leishmania major lipophosphoglycan (LPG), Plasmodium falciparum glycophosphatidylinositol (GPI), zymosan, antigen mixtures from Aspergillus fumigatus, antigen mixtures from Candida albicans, antigen mixtures from measles hemagglutinin, double-stranded RNA, polyadenylic-polyuridylic acid (Poly(A:U)), polyinosine-polycytidylic acid (Poly(I:C)), polyinosine-polycytidylic acid high molecular weight (Poly(I:C) HMW), polyinosine-polycytidylic acid low molecular weight (Poly(I:C) LMW)), LPS from Escherichia coli, LPS from Salmonella species, monophosphoryl lipid A, flagellin, flagellin from B. subtilis, flagellin from P. aeruginosa, flagellin from S. typhimurium, single stranded RNAs with 6UUAU repeats, single stranded RNA homopolymer (ssPolyU naked), HIV-1 LTR-derived ssRNA (ssRNA40), ssRNA with 2 GUCCUUCAA repeats (ssRNA-DR)), imidazoquinoline compounds, imiquimod, Imiquimod VacciGrade™, Gardiquimod VacciGrade™, Gardiquimod™, adenine analog CL264, base analog CL307, guanosine analog loxoribine, TL8-506, thiazoquinoline compound CL075, imidazoquinoline compound CL097, 2Bxy, R848, R848 VacciGrade™, CpG ODN, and Toxoplasma gondii Profilin.

54. The pharmaceutical composition of any one of claims 36-53, wherein the TLR agonist is a TLR7/8 agonist.

55. The pharmaceutical composition of claim 54, wherein the TLR7/8 agonist is R848.

56. The pharmaceutical composition of any one of claims 36-55, wherein the TLR agonist is a TLR5 agonist.

57. The pharmaceutical composition of claim 56, wherein the TLR5 agonist is flagellin.

58. The pharmaceutical composition of any one of claims 36-57, wherein the TLR agonist is a TLR9 agonist.

59. The pharmaceutical composition of claim 58, wherein the TLR9 agonist is a CpG oligonucleotide.

60. The pharmaceutical composition of claim 59, wherein the CpG oligonucleotide comprises or is CpG 1826.

61. The pharmaceutical composition of any of claims 36-60, further comprising administering to the subject an additional adjuvant.

62. The pharmaceutical composition of any one of claims 36-61, wherein the composition comprises an oil in water emulsion.

63. The pharmaceutical composition of any one of claims 36-62, wherein the composition is squalene-based.

64. The pharmaceutical composition of any one of claims 36-63, wherein the composition comprises AddaVax™.

65. The pharmaceutical composition of any one of claims 36-64, wherein the composition further comprises a vaccine composition.

66. The pharmaceutical composition of claim 65, wherein the vaccine composition comprises a flu vaccine, Hepatitis B vaccine, or COVID vaccine.

67. The pharmaceutical composition of claim 66, wherein the vaccine composition comprises or is Fluzone or Heplisav.

68. A method for identifying the efficacy of an adjuvant, the method comprising:
(a) administering the adjuvant to a population of cells;
(b) administering a PRR agonist to the population of cells; and,
(c) measuring expression of one or more cytokines from the cells.

69. The method of claim 68, wherein the one or more cytokines comprise one or more of IL-12p40, IP-10, IL-1β, CCL4, TNF-α, and IFN-β.

70. The method of claim 68, wherein the one or more cytokines comprise IL-12p40, IP-10, IL-1β, CCL4, TNF-α, and IFN-β.

71. The method of any of claims 68-70, wherein the PRR agonist is a TLR agonist.

72. The method of claim 71, wherein the TLR agonist is an agonist to TLR1, TLR2/1, TLR2, TLR2/6, TLR3, TLR4, TLR5 TLR7, TLR8, TLR7/8, TLR9, and/or TLR11.

73. The method of claim 71 or 72, wherein the TLR agonist comprises or consists of one or more of peptidoglycan, triacyl lipoproteins, lipoteichoic acid, peptidoglycan from Bacillus subtilis, peptidoglycan from E. coli 0111: B4, peptidoglycan from Escherichia coli K12, peptidoglycan from Staphylococcus aureus, atypical lipopolysaccharide (LPS), Leptospirosis LPS, Porphyromonas gingivalis LPS, a synthetic diacylated lipoprotein, FSL-1, Pam2CSK4, lipoarabinomannan from M. smegmatis, lipomannan from M. smegmatis; triacylated lipoproteins, Pam3CSK4, MALP-2 from mycoplasma, MALP-404 from mycoplasma, Borrelia burgdorferi OspA, Porin from Neisseria meningitidis, Porin from Haemophilus influenza, Propionibacterium acnes antigen mixtures, Yersinia LcrV, lipomannan from Mycobacterium, lipomannan from Mycobacterium tuberculosis, Trypanosoma cruzi GPI anchor, Schistosoma mansoni lysophosphatidylserine, Leishmania major lipophosphoglycan (LPG), Plasmodium falciparum glycophosphatidylinositol (GPI), zymosan, antigen mixtures from Aspergillus fumigatus, antigen mixtures from Candida albicans, antigen mixtures from measles hemagglutinin, double-stranded RNA, polyadenylic-polyuridylic acid (Poly(A:U)), polyinosine-polycytidylic acid (Poly(I:C)), polyinosine-polycytidylic acid high molecular weight (Poly(I:C) HMW), polyinosine-polycytidylic acid low molecular weight (Poly(I:C) LMW)), LPS from Escherichia coli, LPS from Salmonella species, monophosphoryl lipid A, flagellin, flagellin from B. subtilis, flagellin from P. aeruginosa, flagellin from S. typhimurium, single stranded RNAs with 6UUAU repeats, single stranded RNA homopolymer (ssPolyU naked), HIV-1 LTR-derived ssRNA (ssRNA40), ssRNA with 2 GUCCUUCAA repeats (ssRNA-DR)), imidazoquinoline compounds, imiquimod, Imiquimod VacciGrade™, Gardiquimod VacciGrade™, Gardiquimod™, adenine analog CL264, base analog CL307, guanosine analog loxoribine, TL8-506, thiazoquinoline compound CL075, imidazoquinoline compound CL097, 2Bxy, R848, R848 VacciGrade™, CpG ODN, and Toxoplasma gondii Profilin.

74. The method of claim 71, wherein the TLR agonist is a TLR7/8 agonist.

75. The method of claim 74, wherein the TLR7/8 agonist is R848.

76. The method of claim 71, wherein the TLR agonist is a TLR5 agonist.

77. The method of claim 76, wherein the TLR5 agonist is flagellin.

78. The method of claim 71, wherein the TLR agonist is a TLR9 agonist.

79. The method of claim 78, wherein the TLR9 agonist is a CpG oligonucleotide.
